SEQUENCE LISTING (19 sequences)

SEQ ID NO: 1 - DNA, 270 bp, artificial sequence
  construct encoding rearranged IgVH region for AID-mCherry
acctgcggtgtttatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaagcagctctccctgaagttgagctctgtgaacgccgcggacacggctgtgtattactgtgcgagagttattactagggcgagtcctggcacagacgggaggtac

SEQ ID NO: 2 - protein, 90 aa, artificial sequence
  Synthetic Construct
TCGVYGGSFSGYYWSWIRQPPGKGLEWIGEINHSGSTNYNPSLKSRVTISVDTSKKQLSLKLSSVNAADTAVYYCARVITRASPGTDGRY

SEQ ID NO: 3 - DNA, 270 bp, artificial sequence; identical to SEQ ID NO: 1
  construct encoding rearranged IgVH region for AID-mCherry-CDT1

SEQ ID NO: 4 - protein, 90 aa, artificial sequence; identical to SEQ ID NO: 2
  Synthetic Construct

SEQ ID NO: 5 - DNA, 270 bp, artificial sequence; identical to SEQ ID NO: 1
  construct encoding rearranged IgVH region for AID-mCherry-GEM

SEQ ID NO: 6 - protein, 90 aa, artificial sequence; identical to SEQ ID NO: 2
  Synthetic Construct

SEQ ID NO: 7 - DNA, 29 bp, artificial sequence
  PQL31 primer
atatcaattgagatcccaaatggacagcc

SEQ ID NO: 8 - DNA, 33 bp, artificial sequence
  PQL32 primer
atattctagattacttgtacagctcgtccatgc

SEQ ID NO: 9 - DNA, 35 bp, artificial sequence
  PQL44 primer
tatatgtacaagggatatccatcacactggcggcc

SEQ ID NO: 10 - DNA, 35 bp, artificial sequence
  PQL45 primer
tatatgtacatctagattagatggtgtcctggtcc

SEQ ID NO: 11 - DNA, 34 bp, artificial sequence
  PQL46 primer
tatatgtacatctagattacagcgcctttctccg

SEQ ID NO: 12 - DNA, 31 bp, artificial sequence
  mKO2 FOR primer
atatggatccatcgccaccatggtgagtgtg

SEQ ID NO: 13 - DNA, 31 bp, artificial sequence
  mKO2 REV primer
atatgcggccgccagtgtgatggatatccgc

SEQ ID NO: 14 - DNA, 29 bp, artificial sequence
  F193A FOR primer
cttacgagacgcagctcgtactttgggac

SEQ ID NO: 15 - DNA, 29 bp, artificial sequence
  F193A REV primer
gtcccaaagtacgagctgcgtctcgtaag

SEQ ID NO: 16 - DNA, 21 bp, artificial sequence
  RVHFOR QL primer
tcccaggtgcagctacagcag

SEQ ID NO: 17 - DNA, 21 bp, artificial sequence
  JOL48 QL primer
gtacctgaggagacggtgacc

SEQ ID NO: 18 - DNA, 19 bp, artificial sequence
  2nd round FOR
aggtgcagctacagcagtg

SEQ ID NO: 19 - DNA, 18 bp, artificial sequence
  2nd round REV primer
gccccagacgtccatacc

(19) United States
(12) Patent Application Publication
(10) Pub. No.: US 2016/0369258 A1
(43) Pub. Date: Dec. 22, 2016

(54) RESTRICTING NUCLEAR PROTEIN TO SPECIFIC PHASES OF THE CELL CYCLE

(71) Applicant: UNIVERSITY OF WASHINGTON, Seattle, WA (US)

(72) Inventors: Nancy Maizels, SEATTLE, WA (US); Quy Le, SEATTLE, WA (US)

(73) Assignee: UNIVERSITY OF WASHINGTON, SEATTLE, WA (US)

(21) Appl. No.: 15/122,346

(22) PCT Filed: Mar. 11, 2015
(86) PCT No.: PCT/US15/19990

§ 371 Date: Aug. 29, 2016

Related U.S. Application Data
(60) Provisional application No. 62/094,260, filed on Dec. 19, 2014.
(60) Provisional application No. 61/951,312, filed on Mar. 11, 2014.

(51) Int. Cl. C12N 9/78; C12P 21/00; C12N 15/85; C07K 14/47; C07K 16/00

(52) U.S. Cl. CPC C12N 9/78; C07K 14/4738; C07K 16/00; C12Y 305/04; C12N 15/85; C12P 21/005; C07K 2317/14; C07K 2319/00

(57) ABSTRACT

The present invention relates generally to mutagenesis of target genes that enhances the natural mutagenic capabilities of adaptive immune cells by providing a chimeric construct that exploits the ability of molecules such as AID to stimulate diversification and the ability of a second molecule to restrict nuclear activity of the molecules and/or protect cell viability. The invention provides a method for stimulating diversification in expressed genes, such as antibody genes, using polypeptides whose nuclear activity is restricted to specific phases of the cell cycle. This method can be coupled with selection to identify B cell clones that produce, for example, antibodies of high affinity or specificity, or for developing T cells for immunotherapy. The invention provides an improved means of developing a repertoire of variant immunoglobulins and other polypeptides.

This application claims benefit of U.S. provisional patent application Nos. 61/951,312, filed Mar. 11, 2014, and 62/094,260, filed Dec. 19, 2014, the entire contents of each of which are incorporated by reference into this application.

### STATEMENT REGARDING FEDERALLY SPONSORED RESEARCH

This invention was made with U.S. government support under R01 GM041712, awarded by the National Institutes of Health. The U.S. government has certain rights in the invention.

### TECHNICAL FIELD OF THE INVENTION

The present invention relates generally to constructs and methods that restrict nuclear proteins and polypeptides to specific phases of the cell cycle. One application is in mutagenesis of target genes that enhances the natural mutagenic capabilities of adaptive immune cells by stimulating the process of diversification while protecting the cells from mutagenic factors that can kill cells as they progress through the cell cycle. The invention provides a method for safely initiating mutations and other types of diversification in expressed genes, such as antibody genes. This method can be coupled with selection to identify B cell clones that produce, for example, antibodies of high affinity or specificity. The diversification process can also be used to produce optimized T cells that express chimeric antigen receptors for use in therapeutic applications. The invention thus provides a means of developing a repertoire of variant immunoglobulins and other polypeptides.

### BACKGROUND OF THE INVENTION

Antibodies are molecules that provide a key defense against infection in humans. They are used as therapeutics in treatment of a variety of diseases, from infectious disease to cancer. They are also used as diagnostic reagents in a huge variety of tests carried out daily in clinical and research laboratories.

Antibody specificity and affinity are modified in vivo by processes of mutation, targeted to specific regions within the genes that encode antibodies. Variability in the V region primary sequence (and hence three-dimensional structure and antigen specificity) is the result of processes which alter V region sequence by causing irreversible genetic changes. These changes are programmed during B cell development, and can also be induced in the body in response to environmental signals that activate B cells. Several genetic mechanisms contribute to this variability. Two subpathways of the same mechanism lead to two different mutagenic outcomes, referred to as somatic hypermutation and gene conversion (reviewed (Maizels, 2005)). Somatic hypermutation inserts point mutations. Somatic hypermutation provides the advantage of enabling essentially any mutation to be produced, so a collection of mutated V regions has essentially sampled a large variety of possible mutations.

Activation-induced cytosine deaminase (AID) initiates immunoglobulin (Ig) gene diversification in activated B cells by deaminating C to U (1, 2). This triggers error-prone repair leading to somatic hypermutation (SHM), class switch recombination (CSR) and gene conversion (3-8), and to the chromosomal translocations characteristic of B cell malignancies (9, 10). AID also participates in erasing CpG methylation to reprogram the genome in early development (11-15), promotes B cell tolerance (16, 17) and limits autoimmunity (18, 19).

AID is tightly regulated. Increased AID levels stimulate Ig gene diversification, and also promote translocation (20-23). The AID active site is not optimized for catalysis, but mutations that increase catalytic activity not only accelerate Ig gene diversification but also stimulate translocation and compromise cell viability (24). AID deaminates single-stranded DNA, but not RNA (25-30). AID localizes predominately to the cytoplasm but requires access to the nucleus to function, and subcellular localization is regulated by other proteins (7). AID persistence in the nucleus is limited by proteosomal degradation (31, 32) and by CRM1-dependent nuclear export (33-35). Mutation or deletion of the C-terminal region that includes the nuclear export signal (NES) diminishes AID stability and the efficiency of CSR, and compromises cell viability (36-38). There remains a need for improved methods of stimulating gene diversification, and for methods that can exploit the diversification-enhancing capabilities of AID without compromising cell viability.

### SUMMARY OF THE INVENTION

The invention meets these needs and others by providing materials and methods for restricting nuclear activity of a polypeptide to G1 or to S-G2/M phase of the cell cycle. In one embodiment, the method comprises restricting expression of an enzyme to G1 or to S-G2/M phase of the cell cycle in a host cell. In one embodiment, the enzyme whose expression or nuclear activity is restricted is an enzyme that modifies the sequence and/or structure of a nucleic acid. In one embodiment, the enzyme is AID. In another embodiment, the AID is a catalytically inactive derivative of AID. One example of a catalytically inactive variant of AID is AID H56A. Thus, a representative example of a fusion construct is one that encodes AID^{H56A,F193A}-CDT1. In another embodiment, the enzyme is CRISPR/Cas9 or CRISPR/Cas9^{D10A}.

In one embodiment, the method comprises transfecting a host cell with a fusion construct comprising a nucleotide sequence that expresses the polypeptide fused to a nucleotide sequence that expresses CDT1 or geminin (GEM), wherein a fusion construct expressing CDT1 restricts expression of the enzyme to G1 and a fusion construct expressing GEM restricts expression of the enzyme to S/G2-M phase (Sakaue-Sawano et al. 2008. Cell 132:487).

Additional variations for restricting expression to particular phases of the cell cycle are contemplated. For example, fragments from RAG2 (Li et al. 1996. Immunity 5: 575) for G1 restriction; and Cyclins can be used for cell cycle restricted expression. In some embodiments, the nucleotide sequence that expresses CDT1 or GEM is positioned downstream of the nucleotide sequence that expresses the polypeptide whose nuclear activity is to be restricted.

The invention additionally provides a method of diversification of target sequences while protecting cell viability. The invention provides a cell, which in one embodiment is a lymphocyte, such as a B cell or T cell, modified to enhance diversification of a target gene. The cell comprises a construct as described herein and a target gene of interest. The B cell can be a chicken DT40 B cell or other vertebrate B cell, with a human B cell or a chicken DT40 B cell containing humanized immunoglobulin (Ig) genes (in which human IgH and IgL replace chicken IgH and IgL) preferred for some embodiments.

In one embodiment, the invention provides a nucleic acid construct that expresses a fusion of nuclear export deficient enzyme that initiates or enhances diversification and a polypeptide targeted for cell cycle-dependent nuclear destruction (a “fusion construct”). One representative example of an enzyme that initiates or enhances diversification is a deaminase. Deamination accelerates mutagenesis. In one embodiment, the construct comprises a first nucleotide sequence that expresses activation-induced cytosine deaminase (AID), wherein the AID is modified to prevent nuclear export; and a second nucleotide sequence that expresses chromatin licensing and DNA replication factor 1 (CDT1) or another polypeptide targeted for cell cycle-dependent nuclear destruction, wherein the second nucleotide sequence is operably linked to and downstream of the first nucleotide sequence. AID is a B cell-specific DNA deaminase that initiates Ig gene diversification.

Mutants that promote AID accumulation in the nucleus include, but are not limited to: AID^{F198A} (McBride et al. 2004. J Exp Med 199:1235); AID^{196X} and other C-terminal deletion mutants that remove the nuclear export signal (see, e.g., Ito et al. 2004. PNAS 101: 1975); AID^{F193A}, F193E, F193H, L196A (Geisberger et al. 2009. PNAS 106:6736); and L198S (Patenaude et al. 2009, NSMB 16:17).

Fragments of other proteins that are targeted for nuclear destruction in specific phases of cell cycle can function analogously to the CDT1 tag (Sakaue-Sawano et al. 2008. Cell 132:487) that is exemplified herein to target proteolysis to a fusion protein. These include but are not limited to fragments from: Geminin (Sakaue-Sawano et al. 2008. Cell 132:487): S/G2-M restriction; RAG2 (Li et al. 1996. Immunity 5: 575): G1 restriction; and Cyclins.

The invention provides an adaptive immune cell, such as a B cell or a T cell. A typical example of a B cell for use in the invention is a Ramos human B cell. The B cell can be a human B cell, or a chicken B cell such as DT40, or other vertebrate B cell, or a B cell that has been humanized by replacement of endogenous IgH and IgL genes with human IgH and IgL genes. A typical example of a T cell for use with the invention is a chimeric antigen receptor (CAR) T cell. Candidate lymphocytes for use in the invention are those which can benefit from modulation of the affinity and/or specificity of the cell for its target.

The lymphocyte can be from any vertebrate species. In a typical embodiment, the lymphocyte is from a mammalian or avian species, and in one embodiment, the lymphocyte is a human B cell or human T cell. Other (non-lymphocyte) host cells are suitable for use with the invention as well. In one embodiment, the invention provides a yeast or bacterial cell transfected with the nucleic acid construct.

Typically, the target gene comprises a promoter and a coding region. The coding region of the target gene in the lymphocyte of the invention can be one that encodes any protein or peptide of interest, and need not comprise a complete coding region. In some embodiments, a particular region or domain is targeted for diversification, and the coding region may optionally encode only a portion that includes the region or domain of interest.

In one embodiment, the target gene comprises an immunoglobulin (Ig) gene, wherein the Ig gene comprises an Ig gene enhancer and coding region. The Ig gene can be all or part of an IgL and/or IgH gene. The coding region can be native to the Ig gene, or a heterologous gene. In some embodiments, the target gene is or contains a non-Ig target domain for diversification, as well as domains permitting display of the gene product on the B cell surface, including a transmembrane domain and a cytoplasmic tail.

In one embodiment, the invention provides a method of producing a repertoire of polypeptides having variant sequences of a polypeptide of interest. In one embodiment, the method comprises culturing a lymphocyte transfected with a nucleic acid construct of the invention in conditions that allow expression of the nucleic acid construct. The lymphocyte contains the coding region of the polypeptide of interest, thereby permitting diversification of the coding region. The method further comprises maintaining the culture under conditions that permit proliferation of the lymphocyte until a plurality of lymphocytes and the desired repertoire is obtained. The method optionally further comprises selecting lymphocytes that express a polypeptide exhibiting desired characteristics. For example, a cell expressing an enzyme modified to metabolize an otherwise toxic compound can be selected by growth in a medium containing that compound. Alternatively, a cell that expresses a cytoplasmic fluorescent protein with enhanced fluorescence can be selected by flow for cells with higher mean fluorescent intensity than the starting population. As another example, a cell that expresses a steroid hormone receptor with higher affinity for the hormone can be selected by a fluorescence based assay for increased activity, and a cell that expresses a signaling molecule with higher affinity for a small molecule can be selected by a fluorescence-based signaling assay or other form of such assay that is not toxic to the cell. Likewise, a cell that expresses a DNA damage repair protein with increased activity can be selected for the ability to survive damage by that agent.

In another embodiment, the invention provides a method of producing lymphocytes that produce an optimized polypeptide of interest. In one embodiment, the method comprises culturing a lymphocyte transfected with a nucleic acid construct of the invention in conditions that allow expression of the nucleic acid construct, wherein the lymphocyte contains the coding region of the polypeptide of interest, and wherein and the lymphocyte expresses the polypeptide of interest on the surface of the lymphocyte. The method further comprises selecting cells from the culture that bind a ligand that specifically binds the polypeptide of interest expressed on the lymphocyte surface; and repeating these two steps until cells are selected that have a desired affinity and/or specificity for the ligand that specifically binds the polypeptide of interest. In one embodiment, the polypeptide of interest is an Ig. In a typical embodiment, the Ig is an IgL, IgH or both.

The invention provides a method of producing a repertoire of polypeptides having variant sequences of a polypeptide of interest via diversification of polynucleotide sequences that encode the polypeptide. The cell to be used in the method comprises both the nucleic acid construct of the invention and a nucleic acid encoding the polypeptide of interest. Typically, the method comprises culturing the cell of the invention in conditions that allow expression of the nucleic acids, wherein the target gene contains the coding region of the polypeptide of interest, thereby permitting diversification of the coding region. The method can further comprise maintaining the culture under conditions that permit proliferation of the cell until a plurality of variant polypeptides and the desired repertoire is obtained. The repertoire can then be used for selection of polypeptides having desired properties.

Also provided is a kit that can be used to carry out the methods of the invention. The kit comprises a lymphocyte or other cell of the invention and one or more fusion constructs described herein. The kit further comprises one or more containers, with one or more fusion constructs stored in the containers. Each fusion construct comprises a polynucleotide that can be expressed in the cell. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for use. Directions and or other information can also be included on an insert which is included with the kit.

### BRIEF DESCRIPTION OF THE FIGURES

FIGS. 1A-1E. Images and graphs demonstrating that nuclear AID is degraded more slowly in G1 phase than S-G2/M phases. (FIG. 1A) Representative examples of Ramos cells as analyzed by HCS, with whole cell boundary defined by HCS CellMask, yellow line; and nuclear boundary by DAPI, blue line. Typically, AID is cytoplasmic (N/C<1), but treatment with LMB inhibits nuclear export of AID (N/C>1). In the examples shown, N/C=0.80 (untreated) and 1.34 (0.5 hr LMB). (FIG. 1B) Scatter plots of nuclear vs. cytoplasmic AID-mCherry signals for untreated cells or cells treated with MG132, LMB, or LMB+MG132 as indicated. (FIG. 1C) Quantification of nuclear and cytoplasmic AID-mCherry signal and N/C ratio, relative to untreated cells, at indicated times post-treatment with MG132, LMB, or both. This experiment was repeated 3 times for LMB treatment, and once for MG132 and LMB+MG132 treatment. Dotted line represents no change (fold change of 1). Each point represents a population average, and black bars represent SEM of the population, which are too small to discern. (FIG. 1D) Representative analysis of kinetics of response of AID-mCherry nuclear (solid lines) and cytoplasmic (dashed lines) signals to treatment with MG132, LMB or LMB+MG132 in G1, S and G2/M phase cells. Data presented as in FIG. 1B. (FIG. 1E) Relative rates of nuclear degradation of AID-mCherry in LMB-treated cells in G1, S and G2/M phases. Rates were estimated as the slope of the line defined by the population averages at 1 and 2 hr of treatment, in 4 independent experiments (see FIG. 2A and FIG. 6). Values are presented relative to the slope in G1 phase. SEM, black bars. Significance (p values) shown above graph were determined by two-tailed, unpaired Student's t-test, assuming unequal variances.

FIGS. 2A-2G. Images and graphs that demonstrate that AID-mCherry CDT1 reduces viability and accelerates Ig gene diversification. (FIG. 2A) Flow cytometry of indicated Ramos transductants, showing cell number relative to DNA content and percent of cells in G1 or S-G2/M phases (above), and mCherry signal and fraction of population in each quadrant (below). (FIG. 2B) Representative fluorescence images of indicated transductants, showing mCherry, DAPI and merged signals. (FIG. 2C) Quantification of total, cytoplasmic and nuclear mCherry signals for indicated transductant populations as determined by HCS microscopy, showing the population average and SEM. ***, p<10-10 as determined by two-tailed, unpaired Student's t-test, assuming unequal variances. (FIG. 2D) Nuclear mCherry (arbitrary units) signal in G1, S and G2/M phase cells in indicated transductant populations. Data presented and analyzed as in FIG. 2C. (FIG. 2E) Representative counts of viable cells for indicated transductants at days 3, 7, and 11 after sorting recent transductants for mCherry+. (FIG. 2F) Percentage of sIgM− cells at day 7 after sorting recent transductants for mCherry+ cells; average from 4 independent experiments. **, p<0.005 as determined by two-tailed, unpaired Student's t-test, assuming unequal variances. (FIG. 2G) Percentage of IgG1+ cells in cultures of indicated primary murine B cell transductants at day 4 of in vitro stimulation. *, p<0.05 as determined by two-tailed, unpaired Student's t-test, assuming unequal variances.

FIGS. 3A-3C. Diagrams illustrating frequencies and spectra of mutations at rearranged IgVH regions. (FIG. 3A) Pie charts of hypermutation per IgVH region for indicated Ramos B cell transductants, showing numbers of sequences analyzed (center) and proportions sequences exhibiting 0, 1, 2, 3, 4, ≧5 mutations. Statistical significance determined by χ2 test using data from AID-mCherry transductants as expected values. (FIG. 3B) Genealogies of mutants in transductant populations, based on sequences of VH regions (FIG. 12) including only sequences with distinct mutation spectra. Circles indicate total numbers of point mutations, color-coded as above. (FIG. 3C) Mutation spectra of indicated transductants, showing percentage of each possible single nucleotide substitution among all point mutations, with percentage of all point mutations that occur at each nucleotide shown on the right.

FIGS. 4A-4G. Graphs and images demonstrating elevated nuclear AID is tolerated in G1 phase but toxic in S-G2/M phase. (FIG. 4A) Flow cytometry of indicated Ramos transductants, showing cell number relative to DNA content and percent of cells in G1 or S-G2/M phases (above), and mCherry signal and fraction of population in each quadrant (below). (FIG. 4B) Representative fluorescence images of indicated transductants, showing mCherry, DAPI and merged signals. (FIG. 4C) Quantification of total, cytoplasmic and nuclear mCherry signals by HCS microscopy for indicated transductant populations, showing population average and SEM. Nuclear signals as determined by HCS were corrected for cytoplasmic baseline (see Materials and Methods). ***, p<10-10 as determined by two-tailed, unpaired Student's t-test, assuming unequal variances. (FIG. 4D) Nuclear mCherry (arbitrary units) signal in G1, S and G2/M phase cells in indicated transductant populations. Population average and SEM of a representative experiment are shown. ***, p<10-10 as determined by two-tailed, unpaired Student's t-test, assuming unequal variances. (FIG. 4E) Representative counts of viable cells for indicated transductants at days 3, 7, and 11 after sorting recent transductants for mCherry+ cells (see also FIG. 10). (FIG. 4F) Percentage of sIgM− cells at day 7 after sorting recent transductants for mCherry+ cells. (FIG. 4G) Percentage of IgG1+ cells in cultures of indicated primary murine B cell transductants at day 5 of in vitro stimulation. *, p<0.05 as determined by two-tailed, unpaired Student's t-test, assuming unequal variances.

FIG. 5. Bar graphs illustrating that AID undergoes ubiquitin-dependent proteolysis in the nucleus. Population average of mCherry signal in the nuclear (left) and cytoplasmic (right) compartments are shown at indicated times post-treatment with MG132, LMB, or both. Error bars denote SEM of the population.

FIG. 6. Line graphs demonstrating that LMB treatment causes nuclear accumulation of AID-mCherry, AID-mCherry-CDT1 and AID-mCherry-GEM. Nuclear mCherry signal (relative to untreated cells) as determined by HCS analysis of Ramos AID-Cherry, AID-mCherry-CDT1 and AID-mCherry-GEM transductants treated with LMB for indicated time. Signal shown was determined directly by HCS, and not corrected for cytoplasmic baseline (see Methods).

FIGS. 7A-7B. Data demonstrating that CDT1 and GEM tags confer cell cycle-dependent restriction of nuclear stability to fluorescent reporter proteins. (FIG. 7A) Flow cytometry of Ramos mKO2-CDT1 and mAG-GEM transductants, showing cell number relative to DNA content and percent of cells in G1 or S-G2/M phases (above), and mKO2 signal and fraction of population in each quadrant (below). (FIG. 7B) Representative fluorescence images of Ramos mKO2-CDT1 and Ramos mAG-GEM transductants, showing mKO2 or mAG, DAPI and merged signals.

FIG. 8. Line graphs demonstrating destabilization and redistribution of AID-mCherry, AID-mCherry-CDT1, and AID-mCherry-GEM upon treatment with MG132, LMB, or both. Quantification of nuclear and cytoplasmic AID-mCherry signal and N/C ratio in treated relative to untreated cell populations at indicated times post-treatment with MG132, LMB, or both in Ramos B cells expressing AID-mCherry, AID-mCherry-CDT1, or AIDmCherry-GEM. Each point on the graph represents the population average, and black bars are SEM of the population.

FIGS. 9A-9B. Line and bar graphs illustrating quantification of cell viability of AIDF193A-mCherry, AIDF193A-mCherry-CDT1 and AIDF193A-mCherry-GEM transductants. (FIG. 9A) Cell viability of indicated transductant populations, as determined by trypan blue exclusion. These independent populations were cultured at lower (Expt. a) and higher (Expt. b) density than the experiment shown in the text (FIG. 4E), to ensure that cell density did not account for differences in relative viability. Viability was determined at the indicated day after sorting mCherry+ cells among recent transductants. (FIG. 9B) Cell viability of indicated transductant populations, as determined by assaying ATP levels at days 7 and 11 post-sorting mCherry+ cells among recent Ramos transductants. Viability of the population shown was also analyzed by trypan blue exclusion, and those in Expt. b in FIG. 9A, above.

FIGS. 10A-10C. Data from sIgM loss assays (FIG. 10A) sIgM loss assays of Ramos AID-mCherry, AID-mCherry-CDT1, AID-mCherry-GEM transductants. Shown are representative FACS profiles of Ramos AID-mCherry, AID-mCherry-CDT1, AID-mCherry-GEM and mock transductants at day after sorting mCherry+ cells among recent transductants. Above, mCherry signal gated relative to mock transductants, indicating percentage of mCherry+ cells. Below, sIgM staining profiles, from gate shown above, of mCherry+ cells for AID-mCherry, AID-mCherry-CDT1, and AID-mCherry-GEM transductants; and of mCherry− cells for mock transductants. Percentage of sIgM− cells is shown. (FIG. 10B) Flow cytometry of indicated transductants, showing cell number relative to DNA content and percent of cells in G1 or S-G2/M phases (above), and mKO2 signal and fraction of population in each quadrant (below). (FIG. 10C) Representative FACS profiles of AID-mKO2-CDT1, AID-mKO2-GEM and mock transductants at day 7 after sorting recent transductants for mKO2+ cells. Above, mKO2 signal gated relative to mock transductants, indicating percentage of mKO2+ cells. Below, sIgM staining profiles, from gate shown above, of mKO2+ cells for AID-mKO2-CDT1 and AID-mKO2-GEM transductants; and of mKO2− cells for mock transductants. Percentage of sIgM− cells is shown.

FIGS. 11A-11C. Data showing that AID-mCherry CDT1 accelerates CSR in primary murine B cells. (FIG. 11A) Expression level of AID-mCherry transductants showing MFIs of mock transductants and mCherry+ cells among AID-mCherry transductants. (FIG. 11B) Flow cytometry of indicated transductants of primary murine splenic B cells, showing percent of cells that are mCherry+(above) and fraction of IgG1+ cells among mCherry+ cells (below) at day 4 post transduction. (FIG. 11C) Flow cytometry of indicated transductants of primary murine splenic B cells, showing percent of cells that are mCherry+(above) and fraction of IgG1+ cells among mCherry+ cells (below) at day 5 post transduction.

FIGS. 12A-12C. Sequence analysis of rearranged IgVH regions in single cells for AID-mCherry (FIG. 12A), AID-mCherry-CDT1 (FIG. 12B), and AID-mCherry-GEM (FIG. 12C). The parental nucleic acid sequence is shown in the central line (SEQ ID NOs: 1, 3, and 5, respectively), with positions of nucleotides numbered starting from the first base of first codon, corresponding amino acids (SEQ ID NOs: 2, 4, and 6, respectively) are shown below each codon, and CDR1 and CDR2 underlined. Above the parental sequence, point mutations are indicated as upper case letters, deletions as black bars and insertions as open triangles. Only sequences with unique mutation spectrum are shown.

FIG. 13. Bar graph depicting relative amounts of mutations in VH regions as percent of point mutations, deletions, and insertions in mutated VH regions of AID-mCherry, AID-mCherry-CDT1, or AID-mCherry-GEM transductants.

FIGS. 14A-14B. Images and plot files illustrating analysis of nuclear AID-mCherry signals by confocal microscopy. (FIG. 14A) Fluorescence images of AID-mCherry transductants acquired by confocal fluorescent microscopy. DAPI (left), mCherry (middle) and merge (right) signals are shown. (FIG. 14B) Representative individual AID-mCherry transductants (1-4 in image on left) and plot files of their mCherry fluorescence intensities along arbitrary lines as indicated. Note the range of maximum fluorescence intensities.

FIG. 15. Correction of HCS nuclear signal correction for the contribution of cytoplasmic signal. Scatter plot of nuclear vs. cytoplasmic mCherry signals of Ramos AID-mCherry transductants. Dashed line represents the linear model obtained from linear regression analysis. Right, the equation for the linear model is shown. Nuclear signals as determined by HCS were corrected for cytoplasmic baseline using the formula shown (see Materials and Methods in Example 1).

FIGS. 16A-16E. Graphs depicting HCS assessment of DNA content; nuclear, cytoplasmic and whole cell area and total and average signals in G1, S and G2/M phase Ramos B cell AID-mCherry transductants. (FIG. 16A) Representative cell cycle profile for untreated Ramos B cell AID-mCherry transductant populations, showing fractions identified as G1, S, and G2/M populations. Cell cycle phase was determined based on DNA content as measured by total intensity of DAPI staining. Cells were ranked based on DNA content, and ranks 1-4 assigned to G1 phase, ranks 10-16 to S phase, and ranks 21-24 to G2/M phase. (FIG. 16B) Total intensity of mCherry signal per cell across DNA content. Error bars denote SEM of the population. (FIG. 16C) Average nuclear, cytoplasmic, and whole cell area for G1, S and G2/M phase Ramos B cell AID-mCherry transductant populations. Error bars denote SEM of the population and in some cases are too small to discern clearly. (FIG. 16D) Population average of total intensity of mCherry signal in the nuclear and cytoplasmic compartments and whole cells are shown for G1, S and G2/M phase in Ramos B cell AID-mCherry transductants. Error bars denote SEM of the population and in some cases are too small to discern clearly. (FIG. 16E) Population average of the average intensity of AID-mCherry expression in Ramos B cells in the nuclear and cytoplasmic compartments and whole cells are shown for G1, S and G2/M phase cells. Error bars denote SEM of the population and in some cases are too small to discern clearly.

FIG. 17. Cell cycle profile of Ramos B cells is unaltered by treatment with MG132, LMB, or MG132+LMB treatment in Ramos B cells. Representative cell cycle profiles of Ramos B cell AID-mCherry transductants following treatment with MG132, LMB, or MG132+LMB for indicated time. Estimated percentage of cells in G1, S, and G2/M phase (as determined by the Watson Pragmatic computational model in FlowJo) is tabulated below each cell cycle profile.

FIG. 18. Cell cycle and expression profiles of Ramos transductants at days 3 and 7 post sort. Flow cytometry of Ramos AID-mCherry, AID-mCherry-CDT1, AID-mCherry-GEM, AIDF193A-mCherry, AIDF193A-mCherry-CDT1, AIDF193A-mCherry-GEM, and AIDH56A-mCherry (catalytic mutant) transductants, showing cell number relative to DNA content and percent of cells in G1 or S-G2/M phases (left), and mCherry signal and fraction of population in each quadrant (right) for day 3 and day 7 post sort.

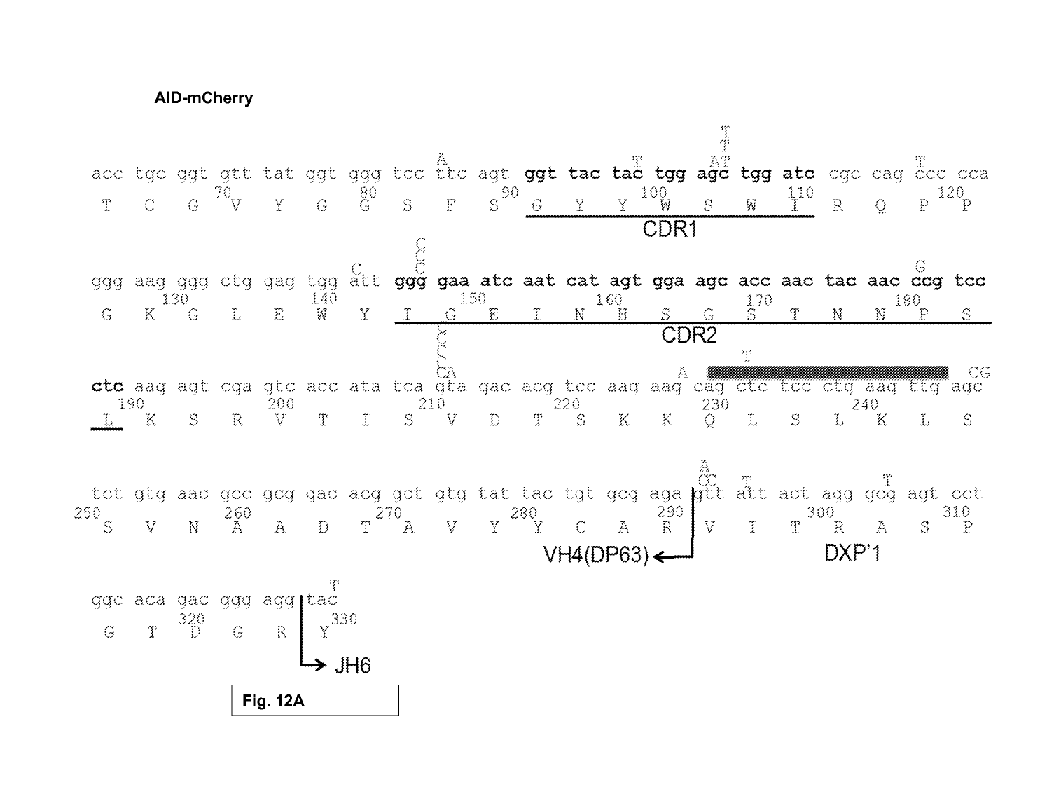

FIGS. 11A-11C, 14B, 16A, 17 and 18 contain cell cycle profile data depicted in graphs that include extremely small text, scatterplots, and other material that may not be decipherable in full detail in the published form of this application. These small text and data points cannot be enlarged by practical means and are not necessary to understand the data conveyed by these figures.

### DETAILED DESCRIPTION OF THE INVENTION

The present invention is based on the unexpected discovery that an enzyme useful for genome engineering can be regulated by fusion of its encoding gene to a protein whose expression is restricted to selected phases of the cell cycle. This allows for an improved method of mutagenesis of target genes by stimulating the process of diversification while protecting the cells from mutagenic factors that can kill cells. The invention provides a method for safely initiating mutations and other types of diversification in expressed genes, such as antibody genes. This method can be coupled with selection to identify B cell clones that produce, for example, antibodies of high affinity or specificity. The diversification process can also be used to produce T cells bearing optimized chimeric antigen receptor for use in therapeutic applications. The invention thus provides a means of developing a repertoire of variant immunoglobulins and other polypeptides.

### DEFINITIONS

All scientific and technical terms used in this application have meanings commonly used in the art unless otherwise specified. As used in this application, the following words or phrases have the meanings specified.

As used herein, “polypeptide” includes proteins, fragments of proteins, and peptides, whether isolated from natural sources, produced by recombinant techniques or chemically synthesized. Peptides of the invention typically comprise at least about 6 amino acids.

As used herein, a “polypeptide targeted for cell cycle-dependent nuclear destruction” means a polypeptide that can target proteolysis to a fusion protein comprising this polypeptide during select phases of the cell cycle. Examples of such polypeptides include fragments of CDT1 (Sakaue-Sawano et al. 2008. Cell 132:487), Geminin (GEM; Sakaue-Sawano et al. 2008. Cell 132:487), RAG2 (Li et al. 1996. Immunity 5: 575), and Cyclins.

As used herein, “CDT1” refers to chromatin licensing and DNA replication factor 1, and includes fragments of CDT1 that can be fused to another polypeptide and that target this fusion protein for degradation in the nucleus during S-G2/M phase of cell cycle.

As used herein, “lymphocyte” refers to adaptive immune cells, including B cells and T cells. A typical example of a B cell for use in the invention is a Ramos human B cell. A typical example of a T cell for use with the invention is a T cell bearing a chimeric antigen receptor (CAR). Candidate lymphocytes for use in the invention are those which can benefit from modulation of the affinity and/or specificity of a cell surface receptor for its target.

As used herein, “nuclear export deficient activation-induced cytosine deaminase (AID)”, means a derivative of the AID protein deficient in nuclear export, such as an AID that lacks a functional nuclear export signal due to one or more mutations at the C terminus or deletion of a portion of the C terminus, including, for example, mutation or deletion of one or more amino acids within the C-terminal residues 183-198, or mutation of another region necessary to enable nuclear export. Examples of nuclear export deficient AIDs include, but are not limited to, AID^{F193A}, AID^{F193E}, AID^{F193H}, AID^{L196A}, AID^{F198A}, AID^{F198S}, AID^{193X} or AID^{196X}. Additional information about AID variants that are deficient in nuclear export can be found in Ito, et al., PNAS 101 (7):1975-1980, 2004; and in Patenaude et al., Nat. Struct. Mol. Biol. 16(5):517-27, 2009.

As used herein, “diversification” of a target gene means a change or mutation in sequence or structure of the target gene. Diversification includes the biological processes of somatic hypermutation, gene conversion, and class switch recombination, which can result in point mutation, templated mutation, DNA deletion and DNA insertion. The diversification factors of the invention can induce, enhance or regulate any of these methods of diversification.

A “mutation” is an alteration of a polynucleotide sequence, characterized either by an alteration in one or more nucleotide bases, or by an insertion of one or more nucleotides into the sequence, or by a deletion of one or more nucleotides from the sequence, or a combination of these.

As used herein, “promoter” means a region of DNA, generally upstream (5′) of a coding region, which controls at least in part the initiation and level of transcription. Reference herein to a “promoter” is to be taken in its broadest context and includes the transcriptional regulatory sequences of a classical genomic gene, including a TATA box or a non-TATA box promoter, as well as additional regulatory elements (i.e., activating sequences, enhancers and silencers) that alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene, although they may also be many kb away. Promoters may contain additional specific regulatory elements, located more distal to the start site to further enhance expression in a cell, and/or to alter the timing or inducibility of expression of a structural gene to which it is operably connected.

As used herein, “operably connected” or “operably linked” and the like means that the polynucleotide elements are linked in a functional relationship. For instance, a promoter or enhancer is operably linked to a coding sequence if it affects the transcription of the coding sequence. Operably linked means that the relevant nucleic acid sequences are typically contiguous and, where necessary to join two protein coding regions, contiguous and in reading frame. “Operably linking” a promoter to a transcribable polynucleotide means placing the transcribable polynucleotide (e.g., protein encoding polynucleotide or other transcript) under the regulatory control of a promoter, which then controls the transcription and optionally translation of that polynucleotide.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “prevent” means to reduce, hinder, or otherwise minimize the occurrence of an event.

As used herein, “a” or “an” means at least one, unless clearly indicated otherwise.

### Fusion Constructs

The invention provides a nucleic acid construct that expresses a fusion of an enzyme that modifies the sequence or structure of DNA or RNA when localized to the nucleus, and a polypeptide targeted for cell cycle-dependent nuclear destruction (a “fusion construct”). In one embodiment, the enzyme is a nuclear export deficient enzyme that initiates or enhances diversification. One representative example of an enzyme that initiates or enhances diversification is a deaminase. Deamination accelerates mutagenesis. In one embodiment, the construct comprises a first nucleotide sequence that expresses activation-induced cytosine deaminase (AID), wherein the AID is modified to prevent nuclear export; and a second nucleotide sequence that expresses chromatin licensing and DNA replication factor 1 (CDT1) or another polypeptide targeted for cell cycle-dependent nuclear destruction, wherein the second nucleotide sequence is operably linked to and downstream of the first nucleotide sequence. AID is a B cell-specific DNA deaminase that initiates Ig gene diversification.

Mutants that prevent AID nuclear export include, but are not limited to: AID^{F198A}(McBride et al. 2004. J Exp Med 199:1235); AID^{196X} and other C-terminal deletion mutants that remove the nuclear export signal (see, e.g., Ito et al. 2004. PNAS 101: 1975); AID^{F193A}, F193E, F193H, L196A (Geisberger et al. 2009. PNAS 106:6736); and L198S (Patenaude et al. 2009, NSMB 16:17).

Fragments of other proteins that are targeted for nuclear destruction in specific phases of cell cycle can function analogously to the CDT1 tag (Sakaue-Sawano et al. 2008. Cell 132:487) that is exemplified herein to target proteolysis to a fusion protein. These include but are not limited to fragments from: Geminin (Sakaue-Sawano et al. 2008. Cell 132:487): S/G2-M restriction; RAG2 (Li et al. 1996. Immunity 5: 575): G1 restriction; and Cyclins.

AID has been fused to a variety of tags to regulate its stability or to visualize it by flow, microscopy, and western blotting. Representative examples of such tags, or fusion partners, include CDT1, GEM, mK02, mAG, GFP, mCherry and T7 tags. Fusion constructs of the invention may optionally include a tag to facilitate visualization, detection, or tracking.

Fusion constructs may generally be prepared using standard techniques. For example, DNA sequences encoding the peptide components may be assembled separately, and ligated into an appropriate expression vector. The ligated DNA sequences are operably linked to suitable transcriptional or translational regulatory elements. The 3′ end of the DNA sequence encoding one peptide component is ligated, with or without a linker, to the 5′ end of a DNA sequence encoding the second peptide component so that the reading frames of the sequences are in phase. This permits translation into a single fusion protein that retains the biological activity of both component peptides. Additional fusion partners, or visualization tags, may be joined in a similar manner. Thus, a fusion construct of the invention optionally further comprises a detectable marker. In one embodiment, the detectable marker is a fluorescent protein.

A peptide linker sequence may be employed to separate the first and the second peptide components by a distance sufficient to ensure that each peptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional regions on the first and second peptides; and (3) the lack of hydrophobic or charged residues that might react with the peptide functional regions. Preferred peptide linker sequences contain Gly, Asn and Ser residues. Other near neutral amino acids, such as Thr and Ala may also be used in the linker sequence.

### Lymphocytes and Other Host Cells

The invention provides an adaptive immune cell, such as a B cell or a T cell. A typical example of a B cell for use in the invention is a Ramos human B cell. The B cell can be a human B cell, or a chicken B cell such as DT40, or other vertebrate B cell, or a B cell that has been humanized by replacement of endogenous IgH and IgL genes with human IgH and IgL genes. A typical example of a T cell for use with the invention is a chimeric antigen receptor (CAR) T cell. Candidate lymphocytes for use in the invention are those which can benefit from modulation of the affinity and/or specificity of the cell for its target.

The lymphocyte can be from any vertebrate species. In a typical embodiment, the lymphocyte is from a mammalian or avian species, and in one embodiment, the lymphocyte is a human B cell or human T cell. Other (non-lymphocyte) host cells are suitable for use with the invention as well. In one embodiment, the invention provides a yeast or bacterial cell transfected with the nucleic acid construct.

B cells are natural producers of antibodies, making them an attractive cell for production of both improved antibodies and improved non-immunoglobulin proteins and polypeptides. DT40 B cells are an effective starting point for evolving specific and high affinity antibodies by iterative cycles of hypermutation and selection (Cumbers et al., 2002; Seo et al., 2005). DT40 cells have several advantages over other vehicles tested for this purpose. DT40 constitutively diversifies its Ig genes in culture, and proliferates more rapidly than human B cell lines (10-12 hr generation time, compared to 24 hr); clonal populations can be readily isolated because cells are easily cloned by limiting dilution, without addition of special factors or feeder layers; and DT40 carries out efficient homologous gene targeting (Sale, 2004), so specific loci can be replaced at will allowing one to manipulate factors that regulate hypermutation.

The invention provides a novel platform for generating high affinity antibodies and other optimized polypeptides. In one embodiment, the vehicle for antibody evolution is a B cell line, DT40, which naturally produces antibodies, and which has been engineered to facilitate mutagenesis. Like other B cells, DT40 expresses antibodies on the cell surface, allowing convenient clonal selection for high affinity and optimized specificity, by fluorescence or magnetic-activated cell sorting. In the DT40 cell line, hypermutation is carried out by the same pathway that has been perfected over millions of years of vertebrate evolution to Ig gene hypermutation in a physiological context. This highly conserved pathway targets mutations preferentially (though not exclusively) to the complementarity-determining regions (CDRs), the subdomains of the variable (V) regions that make contact with antigen.

Thus far, the use of DT40 (and other cultured B cell lines) for antibody selection has been limited because the rate of hypermutation is very slow, about 0.1%-1% that of physiological hypermutation. To accelerate hypermutation, key regulatory sites and factors have been manipulated, taking advantage of our current sophisticated understanding of the molecular mechanisms of hypermutation.

Although chicken DT40 B cells offer many advantages, in some embodiments it may be desired to use human B cells. Alternatively, one can employ humanized Ig genes with the chicken DT40 B cells. By humanizing the DT40 immunoglobulin genes, the utility of this platform for therapeutics can be broadened, as the antibodies generated in the DT40 platform could be used directly for treatment.

There is ample documentation of the utility of humanized antibody genes, and a number of validated approaches for humanization, as reviewed recently (Waldmann and Morris, 2006; Almagro and Fransson, 2008). Humanization is effected by substitution of human Ig genes for the chicken Ig genes, and this is readily done in DT40 by taking advantage of the high efficiency of homologous gene targeting. The substitutions are designed to modify distinct parts of the heavy and light chain loci. Substitution could produce DT40 derivatives that generate entirely humanized antibodies, by swapping V(D)J and C regions; or chimeric antibodies (humanized C regions but not V regions). These replacements will not alter the adjacent cis-regulatory elements or affect their ability to accelerate hypermutation. The conserved mechanisms that promote hypermutation will target mutagenesis to the CDRs of humanized sequences. The humanized line can thus be used for accelerated development of human monoclonals in cell culture, providing a dual platform for rapid production of useful antibodies for either therapeutic or diagnostic purposes.

In addition, one can optimize antibody effector function by C region replacement. Antibody-based immunotherapy is a powerful approach for therapy, but this approach thus far been limited in part by availability of specific antibodies with useful effector properties (Hung et al., 2008; Liu et al., 2008). The constant (C) region of an antibody determines effector function. Substitutions of either native or engineered human C regions can be made by homologous gene targeting in the DT40 vehicle to generate antibodies with desired effector function.

### Target Genes

Typically, the target gene comprises a promoter and a coding region. The coding region of the target gene in the lymphocyte of the invention can be one that encodes any protein or peptide of interest, and need not comprise a complete coding region. In some embodiments, a particular region or domain is targeted for diversification, and the coding region may optionally encode only a portion that includes the region or domain of interest.

In one embodiment, the target gene comprises an immunoglobulin (Ig) gene, wherein the Ig gene comprises an Ig gene enhancer and coding region. The Ig gene can be all or part of an IgL and/or IgH gene. The coding region can be native to the Ig gene, or a heterologous gene. In some embodiments, the target gene is or contains a non-Ig target domain for diversification, as well as domains permitting display of the gene product on the B cell surface, including a transmembrane domain and a cytoplasmic tail.

### Methods and Uses of the Invention

The invention provides a method of producing a repertoire of polypeptides having variant sequences of a polypeptide of interest. In one embodiment, the method comprises culturing a lymphocyte transfected with a nucleic acid construct of the invention in conditions that allow expression of the nucleic acid construct. The lymphocyte contains the coding region of the polypeptide of interest, thereby permitting diversification of the coding region. The method further comprises maintaining the culture under conditions that permit proliferation of the lymphocyte until a plurality of lymphocytes and the desired repertoire is obtained. In another embodiment, the invention provides a method of producing lymphocytes that produce an optimized polypeptide of interest.

In one embodiment, the method comprises culturing a lymphocyte transfected with a nucleic acid construct of the invention in conditions that allow expression of the nucleic acid construct, wherein the lymphocyte contains the coding region of the polypeptide of interest, and wherein and the lymphocyte expresses the polypeptide of interest on the surface of the lymphocyte. The method further comprises selecting cells from the culture that bind a ligand that specifically binds the polypeptide of interest expressed on the lymphocyte surface; and repeating these two steps until cells are selected that have a desired affinity and/or specificity for the ligand that specifically binds the polypeptide of interest. In one embodiment, the polypeptide of interest is an Ig. In a typical embodiment, the Ig is an IgL, IgH or both.

The invention provides a method of producing a repertoire of polypeptides having variant sequences of a polypeptide of interest via diversification of polynucleotide sequences that encode the polypeptide. The cell to be used in the method comprises both the nucleic acid construct of the invention and a nucleic acid encoding the polypeptide of interest. Typically, the method comprises culturing the cell of the invention in conditions that allow expression of the nucleic acids, wherein the target gene contains the coding region of the polypeptide of interest, thereby permitting diversification of the coding region. The method can further comprise maintaining the culture under conditions that permit proliferation of the cell until a plurality of variant polypeptides and the desired repertoire is obtained. The repertoire can then be used for selection of polypeptides having desired properties.

In embodiments in which the polypeptide of interest is an Ig, such as an IgL, IgH or both, the ligand may be a polypeptide, produced by recombinant or other means, that represents an antigen. The ligand can be bound to or linked to a solid support to facilitate selection, for example, by magnetic-activated cell selection (MACS). In another example, the ligand can be bound to or linked to a fluorescent tag, to allow for or fluorescence-activated cell sorting (FACS). Those skilled in the art appreciate that other methods of labeling and selecting cells are known and can be used in this method.

The invention also provides a vehicle for selection of T cell receptors. T cell-based immunotherapy has great potential (Blattman and Greenberg, 2004). T cell receptor specificity and affinity is governed by CDR contacts (Chlewicki et al., 2005). Selection for specificity or high affinity T cell receptors can be carried out in a DT40 vehicle, which has been modified by substitution of T cell receptors (V regions or entire genes) for the Ig loci; or directly in human T cells.

Production of catalytic Igs is another aspect of the invention. The Ig-related methods of the invention are not simply limited to the production of Igs for binding and recognition, as the target Ig could also be used for catalysis. After development of a stable molecule that mimics the transition state of an enzymatic reaction, DT40 cells can be used to evolve an antibody that binds and stabilizes the actual chemical transition state. After identifying clones that produce an Ig capable of binding the intermediate, the system can be used again to screen for catalytic activity of Igs on the real substrate in culture. Once some activity has been demonstrated in this system, optimization of activity can proceed by further evolution of the Ig loci through mutagenesis. Thus, invention does not require animal immunization (a slow step), immortalization by hybridoma technology, and the inefficiency of later having to screen hybridomas for antibodies that demonstrate catalytic activity.

The genomic structure at the Ig loci has evolved to promote mutagenesis of 1-1.5 kb downstream of the promoter. This system can be harnessed to mutate short regions of genes. Clonal selection based on surface protein expression can be incorporated by fusion of the region of interest to a portion of a gene expressing elements that mediate surface expression. Exemplary elements for surface expression include a signal peptide, transmembrane domain and cytoplasmic tail from a protein expressed on the B cell surface (Chou et al., 1999; Liao et al., 2001).

The invention can also be used for the production of recognition arrays. The ability to evolve cells harboring receptors with affinities for a large spectrum of antigens allows the development of recognition arrays. Combining this technology with intracellular responses/signaling from receptor stimulation in DT40 (such as measurement of Ca2+ by aequorin (Rider et al., 2003) or use of reporter gene transcription) would create a useful biosensor. Diversified clones would be spotted into arrays or 96 well plates, and exposed to samples. Each sample would yield a “fingerprint” of stimulation. The arrays would permit qualitative comparisons of biological/medical, environmental, and chemical samples. Analysis need not be limited to the analysis of proteins, as is the case for comparative techniques like 2D gels, since all forms of compounds could have antigenic properties. Furthermore, the arrays would lead to the identification of components without knowledge of their presence beforehand.

The invention additionally provides a method of restricting nuclear activity of a polypeptide to G1 or to S-G2/M phase of the cell cycle. In one embodiment, the method comprises restricting expression of an enzyme to G1 or to S-G2/M phase of the cell cycle in a host cell. In one embodiment, the enzyme whose expression or nuclear activity is restricted is AID. In one embodiment, the AID is a catalytically inactive derivative of AID. One example of a catalytically inactive variant of AID is AID H56A. Thus, a representative example of a fusion construct is one that encodes AID^{H56A,F193A}-CDT1. In another embodiment, the enzyme is CRISPR/Cas9 or CRISPR/Cas9^{D10A}.

In one embodiment, the method comprises transfecting a host cell with a fusion construct comprising a nucleotide sequence that expresses the polypeptide fused to a nucleotide sequence that expresses CDT1 or geminin (GEM), wherein a fusion construct expressing CDT1 restricts expression of the enzyme to G1 and a fusion construct expressing GEM restricts expression of the enzyme to S/G2-M phase (Sakaue-Sawano et al. 2008. Cell 132:487).

Additional variations for restricting expression to particular phases of the cell cycle are contemplated. For example, fragments from RAG2 (Li et al. 1996. Immunity 5: 575) for G1 restriction; and Cyclins can be used for cell cycle restricted expression. In some embodiments, the nucleotide sequence that expresses CDT1 or GEM is positioned downstream of the nucleotide sequence that expresses the polypeptide whose nuclear activity is to be restricted.

### Kits

For use in the methods described herein, kits are also within the scope of the invention. Such kits can comprise a carrier, package or container that is compartmentalized to receive one or more containers such as vials, tubes, and the like, each of the container(s) comprising one of the separate elements (e.g., cells, constructs) to be used in the method.

Typically, the kit comprises a lymphocyte or other cell of the invention and one or more fusion constructs described herein. The kit further comprises one or more containers, with one or more fusion constructs stored in the containers. Each fusion construct comprises a polynucleotide that can be expressed in the cell. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for use. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1

### Cell Cycle Regulates Nuclear Stability of AID and the Cellular Response to AID

This example illustrates features that support the invention, namely the means by which a diversification factor like AID can be modified to persist in the nucleus and also coupled with a nuclear destruction signal to protect cell viability. AID (Activation Induced Deaminase) deaminates cytosines in DNA to initiate immunoglobulin gene diversification and to reprogram the genome in early development. This example demonstrates how the cell cycle regulates AID and the cellular response to AID. Using high content screening microscopy to quantify subcellular localization, we show that AID undergoes nuclear degradation more slowly in G1 phase than in S or G2-M phase. Using CDT1 and GEM tags to promote degradation of nuclear AID in specific phases of cell cycle, we show that elevated nuclear AID accelerates somatic hypermutation and class switch recombination. Strikingly, nuclear AID is tolerated in G1 phase but compromises cell viability in other phases of cell cycle. These results establish that cell cycle regulates subcellular localization and nuclear stability of AID, and identify an unexpected connection between spatiotemporal regulation of AID and cell viability

AID levels are constant during cell cycle (31, 36), but several observations suggested that cell cycle may regulate AID. In DT40 chicken B cells, brief treatment with leptomycin B (LMB), an inhibitor of the CRM1-dependent nuclear export, increases nuclear AID signal in G1 phase cells (39); Polη, which copies donor DNA in AID-initiated gene conversion, co-localizes with the diversifying Igλ_{R} allele predominately in G1 phase (40); UNG2 removes uracils produced upon deamination by AID predominately in G1 phase (41); and RPA initially accumulates at Ig switch regions in G1 phase (42).

We have now asked if cell cycle regulates subcellular localization, stability or physiological activity of AID. We demonstrate that nuclear degradation occurs more slowly in G1 phase than in S-G2/M phase cells, and that the presence of AID in the nucleus in G1 phase accelerates SHM and CSR. Strikingly, elevated nuclear AID is tolerated in G1 phase, but it compromises fitness in other stages of cell cycle. These results establish that cell cycle regulates both nuclear AID and the ability of cells to respond to AID.

### Results

Nuclear AID is More Stable in G1 Phase than in S or G21M Phases.

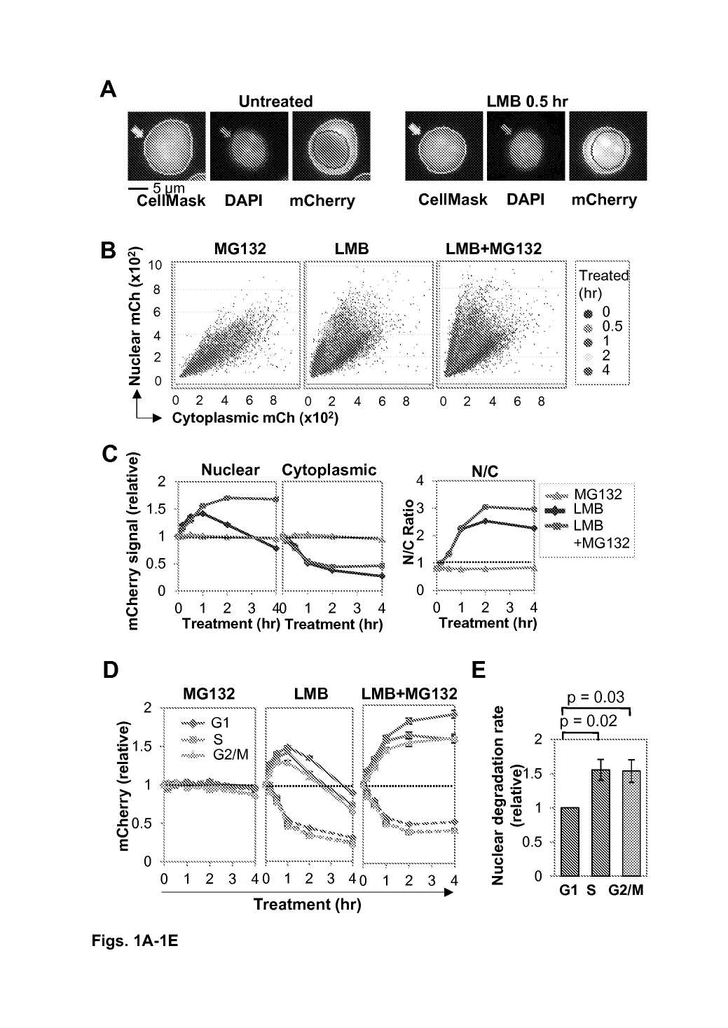

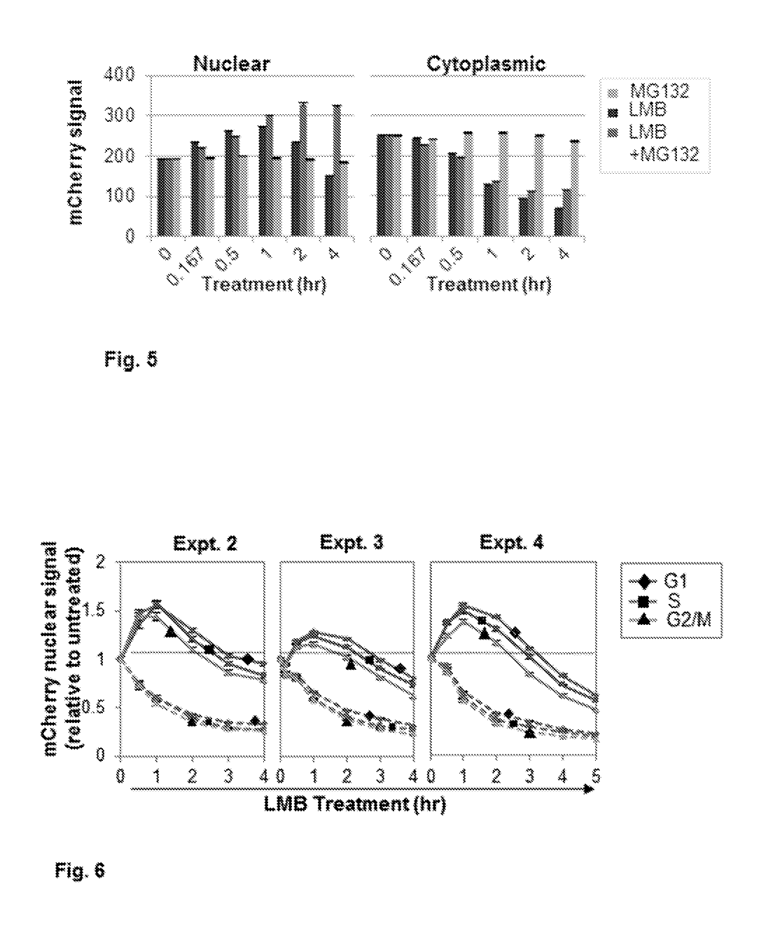

We analyzed subcellular distribution of AID in the human B cell line, Ramos, transduced with a lentiviral construct expressing human AID fused to the mCherry fluorescent protein at the C-terminus. Ramos B cells express endogenous AID and actively diversify their Ig genes, so the pathways that regulate and respond to damage by AID are intact. Cells were analyzed by high content screening (HCS) microscopy (43), a flow-based approach that automatically quantifies signals per unit area (pixels) in each compartment of each cell (FIG. 1A). Nuclear and cytoplasmic signals essentially overlapped in populations that were untreated or treated with MG132, an inhibitor of the ubiquitin-dependent 26S proteasome; while treatment with LMB or both LMB+MG132 rapidly increased nuclear signal in most cells (FIG. 1B). Quantification established that nuclear signal was unaffected by MG132 treatment; rapidly increased (1.5-fold) and then declined in response to LMB treatment; and increased (1.7-fold) and plateaued in response to treatment with both LMB+MG132 (FIG. 1C; FIG. 5, Table 1). The cytoplasmic signal was unaffected by MG132 treatment, but diminished upon treatment with LMB or LMB+MG132, paralleling the increase in nuclear signal. These results are consistent with previous reports that AID undergoes nuclear proteolysis (31, 32).

TABLE 1
Probability Tests for FIG. 1C
AID-mCherry Transductants: Treated vs. Untreated

| | 0 hr | 0.167 hr | | 0.5 hr | | 1 hr | | 2 hr | | 4 hr | |
|---|---|---|---|---|---|---|---|---|---|---|---|
| | N | N | p | N | p | N | p | N | p | N | p |
| LMB | | | | | | | | | | | |
| Nuclear AID-mCherry | 6141 | 3394 | 1.59E−59 | 4752 | 8.55E−185 | 6393 | 1.07E−289 | 3327 | 3.49E−61 | 5023 | 1.17E−130 |
| Cytoplasmic | 6141 | 3394 | 2.30E−02 | 4752 | 1.07E−53 | 6393 | 0.00E+00 | 3327 | 0.00E+00 | 5023 | 0.00E+00 |
| AID-mCherry | | | | | | | | | | | |
| LMB + MG132 | | | | | | | | | | | |
| Nuclear AID-mCherry | 6141 | 5009 | 9.40E−37 | 4682 | 1.09E−132 | 6029 | 0.00E+00 | 1988 | 1.01E−207 | 1918 | 6.78E−168 |
| Cytoplasmic | 6141 | 5009 | 2.08E−16 | 4682 | 2.61E−93 | 6029 | 0.00E+00 | 1988 | 0.00E+00 | 1918 | 0.00E+00 |
| AID-mCherry | | | | | | | | | | | |
| MG132 | | | | | | | | | | | |
| Nuclear AID-mCherry | 6141 | 5569 | 6.80E−01 | 5822 | 4.54E−04 | 3063 | 5.41E−01 | 2862 | 5.14E−01 | 5569 | 9.91E−04 |
| Cytoplasmic | 6141 | 5569 | 2.56E−03 | 5822 | 1.62E−02 | 3063 | 1.27E−02 | 2862 | 7.77E−01 | 5569 | 7.92E−05 |
| AID-mCherry | | | | | | | | | | | |

| | | | | |
|---|---|---|---|---|
| 0.167 hr | 0.5 hr | 1 hr | 2 hr | 4 hr |

AID-mCherry Transductants: LMB vs. LMB + MG132 Treated

| | | | | | |
|---|---|---|---|---|---|
| Nuclear AID-mCherry | 5.37E−08 | 6.21E−08 | 8.52E−25 | 6.89E−99 | 3.54E−265 |
| Cytoplasmic AID-mCherry | 1.75E−07 | 1.84E−05 | 4.49E−10 | 8.17E−22 | 1.59E−118 |

AID-mCherry Transductants: LMB vs. MG132 Treated

| | | | | | |
|---|---|---|---|---|---|
| Nuclear AID-mCherry | 1.34E−51 | 7.10E−141 | 1.38E−275 | 5.13E−53 | 2.13E−59 |
| Cytoplasmic AID-mCherry | 6.01E−01 | 3.89E−80 | 0.00E+00 | 0.00E+00 | 0.00E+00 |

AID-mCherry Transductants: MG132 vs. LMB + MG132 Treated

| | | | | | |
|---|---|---|---|---|---|
| Nuclear AID-mCherry | 7.23E−30 | 1.59E−94 | 0.00E+00 | 8.91E−201 | 1.39E−175 |
| Cytoplasmic AID-mCherry | 3.54E−07 | 2.52E−135 | 0.00E+00 | 0.00E+00 | 6.44E−256 |

Statistical tests were performed using two-tailed, unpaired Student's t-test, assuming unequal variances, for comparison of nuclear and cytoplasmic AID-mCherry signal and the N/C ratio between different treatment groups and between different times post treatment and untreated control in each treatment group.

We used HCS to quantify AID-mCherry subcellular distribution in Ramos B cells in each phase of cell cycle (FIG. 1D). Treatment with MG132 had little effect on nuclear or cytoplasmic AID-mCherry signals in any phase of cell cycle. Treatment with LMB or LMB+MG132 caused the cytoplasmic signal to drop by 50% in all stages of cell cycle, evidence of the importance of nuclear export in maintaining cytoplasmic signal. Treatment with LMB caused the nuclear AID-mCherry signal to increase (0-1 hr) and then drop, while treatment with LMB+MG132 caused this signal to increase and then plateau; thus the drop in nuclear signal following treatment with LMB alone was due to proteolysis. Notably, LMB treatment caused a sharper initial increase and more gradual decrease in nuclear signal in G1 phase than S or G2/M phase cells; while LMB+MG132 treatment resulted in a significantly higher relative signal in G1 phase than S or G2/M phase cells (at 2 hr, G1 vs. S, p=1.4×10⁻³; G1 vs. G2/M, p=1.8×10⁻⁵; FIG. 1D, right, Table 2). Thus, nuclear stability of AID-mCherry is cell cycle dependent, and stability is highest in G1 phase.

TABLE 2A
Probability test for FIG. 1D: Cell Cycle Comparisons

| | Nuclear AID-mCherry | | | Cytoplasmic AID-mCherry | | |
|---|---|---|---|---|---|---|
| | | G1 vs. | S vs. | | G1 vs. | S vs. |
| | G1 vs. S | G2/M | G2/M | G1 vs. S | G2/M | G2/M |
| LMB | | | | | | |
| 0 hr | 1.00E+00 | 1.00E+00 | 1.00E+00 | 1.00E+00 | 1.00E+00 | 1.00E+00 |
| 0.167 hr | 1.58E−01 | 4.44E−04 | 5.08E−02 | 2.68E−01 | 2.01E−03 | 6.89E−02 |
| 0.5 hr | 4.35E−02 | 4.37E−04 | 1.37E−01 | 6.37E−01 | 1.87E−02 | 2.10E−02 |
| 1 hr | 1.08E−02 | 7.08E−13 | 8.72E−06 | 2.12E−03 | 3.01E−11 | 5.81E−04 |
| 2 hr | 6.57E−10 | 5.86E−12 | 2.48E−01 | 4.02E−16 | 2.24E−14 | 8.65E−01 |
| 4 hr | 2.50E−21 | 4.02E−32 | 1.75E−04 | 2.26E−20 | 3.80E−21 | 8.03E−03 |
| LMB + MG132 | | | | | | |
| 0.167 hr | 2.56E−03 | 2.76E−04 | 2.91E−01 | 1.98E−01 | 4.68E−03 | 1.30E−01 |
| 0.5 hr | 1.29E−04 | 1.99E−04 | 6.60E−01 | 8.76E−03 | 3.78E−03 | 5.58E−01 |
| 1 hr | 1.22E−01 | 8.08E−07 | 1.66E−03 | 2.95E−06 | 2.40E−09 | 1.33E−01 |
| 2 hr | 1.42E−03 | 1.83E−05 | 1.49E−01 | 2.24E−11 | 1.39E−06 | 4.50E−01 |
| 4 hr | 1.68E−06 | 1.77E−05 | 8.02E−01 | 3.19E−10 | 2.87E−04 | 8.70E−01 |
| MG132 | | | | | | |
| 0.167 hr | 8.74E−02 | 2.40E−01 | 7.76E−01 | 1.04E−01 | 1.53E−01 | 9.09E−01 |
| 0.5 hr | 6.03E−01 | 6.03E−01 | 9.82E−01 | 3.42E−02 | 3.04E−01 | 3.95E−01 |
| 1 hr | 8.91E−01 | 4.59E−02 | 8.50E−02 | 4.82E−01 | 1.68E−02 | 8.20E−03 |
| 2 hr | 5.11E−02 | 1.56E−01 | 7.65E−01 | 4.90E−03 | 3.95E−04 | 4.10E−01 |
| 4 hr | 6.17E−01 | 1.01E−03 | 4.88E−03 | 8.63E−01 | 5.71E−03 | 5.40E−02 |

TABLE 2B
Probability test for FIG. 1D: Comparisons of Treated to Untreated Cells

| | 0 hr | 0.167 hr | | 0.5 hr | | 1 hr | | 2 hr | | 4 hr | |
|---|---|---|---|---|---|---|---|---|---|---|---|
| Treatment time | N | N | p | N | p | N | p | N | p | N | p |
| LMB | | | | | | | | | | | |
| G1 | | | | | | | | | | | |
| Nuclear AID-mCherry | 1899 | 1112 | 9.08E−30 | 1532 | 9.26E−75 | 2100 | 9.51E−125 | 1022 | 2.09E−41 | 1415 | 1.89E−11 |
| Cytoplasmic AID-mCherry | 1899 | 1112 | 8.66E−01 | 1532 | 1.75E−18 | 2100 | 1.25E−131 | 1022 | 3.02E−181 | 1415 | 5.84E−266 |
| S | | | | | | | | | | | |
| Nuclear AID-mCherry | 1141 | 594 | 3.21E−13 | 844 | 7.38E−33 | 1218 | 7.89E−60 | 634 | 3.08E−07 | 1045 | 2.52E−43 |
| Cytoplasmic AID-mCherry | 1141 | 594 | 3.57E−01 | 844 | 1.67E−09 | 1218 | 7.27E−99 | 634 | 4.58E−159 | 1045 | 4.62E−199 |
| G2/M | | | | | | | | | | | |
| Nuclear AID-mCherry | 934 | 493 | 5.50E−06 | 719 | 1.00E−19 | 949 | 6.39E−24 | 457 | 1.23E−03 | 684 | 1.06E−47 |
| Cytoplasmic AID-mCherry | 934 | 493 | 8.79E−03 | 719 | 9.48E−15 | 949 | 1.00E−84 | 457 | 2.01E−118 | 684 | 1.75E−153 |
| LMB + MG132 | | | | | | | | | | | |
| G1 | | | | | | | | | | | |
| Nuclear AID-mCherry | 1899 | 1558 | 4.38E−20 | 1420 | 8.23E−56 | 1880 | 1.54E−145 | 567 | 1.11E−70 | 422 | 6.70E−54 |
| Cytoplasmic AID-mCherry | 1899 | 1558 | 3.70E−05 | 1420 | 4.32E−24 | 1880 | 3.57E−109 | 567 | 2.12E−130 | 422 | 1.89E−102 |
| S | | | | | | | | | | | |
| Nuclear AID-mCherry | 1141 | 954 | 7.85E−06 | 895 | 5.11E−19 | 1107 | 2.45E−76 | 393 | 2.14E−39 | 411 | 2.31E−29 |
| Cytoplasmic AID-mCherry | 1141 | 954 | 1.69E−05 | 895 | 1.28E−24 | 1107 | 8.92E−92 | 393 | 4.16E−130 | 411 | 1.98E−116 |
| G2/M | | | | | | | | | | | |
| Nuclear AID-mCherry | 934 | 580 | 1.03E−02 | 570 | 1.03E−11 | 811 | 1.04E−42 | 263 | 7.47E−21 | 293 | 1.50E−24 |
| Cytoplasmic AID-mCherry | 934 | 580 | 9.48E−07 | 570 | 3.39E−19 | 811 | 2.54E−73 | 263 | 3.30E−86 | 293 | 1.19E−52 |
| MG132 | | | | | | | | | | | |
| G1 | | | | | | | | | | | |
| Nuclear AID-mCherry | 1899 | 1413 | 1.90E−01 | 1701 | 1.07E−01 | 1860 | 6.52E−01 | 949 | 3.28E−01 | 605 | 2.51E−01 |
| Cytoplasmic AID-mCherry | 1899 | 1413 | 5.23E−01 | 1701 | 1.52E−01 | 1860 | 2.41E−01 | 949 | 4.36E−02 | 605 | 5.27E−02 |
| S | | | | | | | | | | | |
| Nuclear AID-mCherry | 1141 | 782 | 5.19E−01 | 1033 | 4.77E−01 | 1042 | 8.22E−01 | 583 | 2.14E−01 | 616 | 1.06E−01 |
| Cytoplasmic AID-mCherry | 1141 | 782 | 5.08E−02 | 1033 | 5.66E−01 | 1042 | 1.44E−01 | 583 | 2.98E−01 | 616 | 1.34E−01 |
| G2/M | | | | | | | | | | | |
| Nuclear AID-mCherry | 934 | 591 | 8.00E−01 | 791 | 5.32E−01 | 828 | 1.48E−01 | 431 | 4.54E−01 | 436 | 8.76E−06 |
| Cytoplasmic AID-mCherry | 934 | 591 | 1.03E−01 | 791 | 8.28E−01 | 828 | 3.15E−01 | 431 | 8.62E−02 | 436 | 3.11E−05 |

Statistical tests were performed using two-tailed, unpaired Student's t-test, assuming unequal variances, for comparison of nuclear and cytoplasmic AID-mCherry signal and the N/C ratio between G1 and S; G1 and G2/M; and S and G2/M at different times post-treatment in each treatment group.

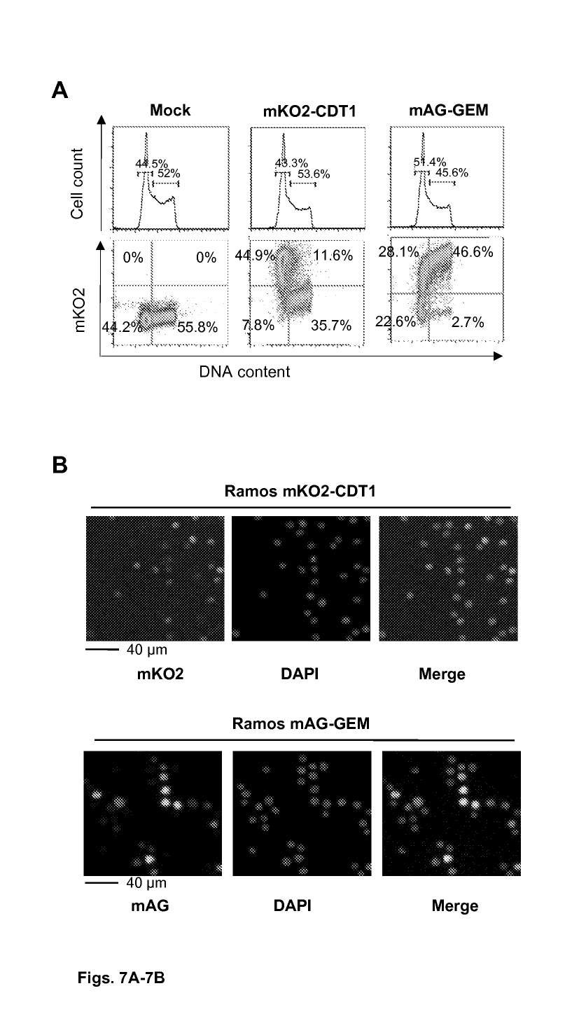

Comparison of the slopes of the LMB response curves between the 1 and 2 hr time points (FIG. 1D, center) suggested that degradation occurred more rapidly in S-G2/M phase than G1 phase. To quantify this, we calculated the average rate of loss of nuclear signal between 1-2 hr of treatment, as defined by the slope of the line between these time points, for 4 independent experiments (FIG. 1D; and FIG. 6). Rates of initial degradation were 1.56-fold and 1.54-fold higher in S and G2/M phases (p=0.02 and 0.03, respectively; FIG. 1E) than in G1 phase. We conclude that nuclear AID-mCherry is degraded more rapidly in S and G2/M phase than in G1 phase.

Elevated Nuclear AID Compromises Viability of AID-mCherry-CDT1 Transductants.

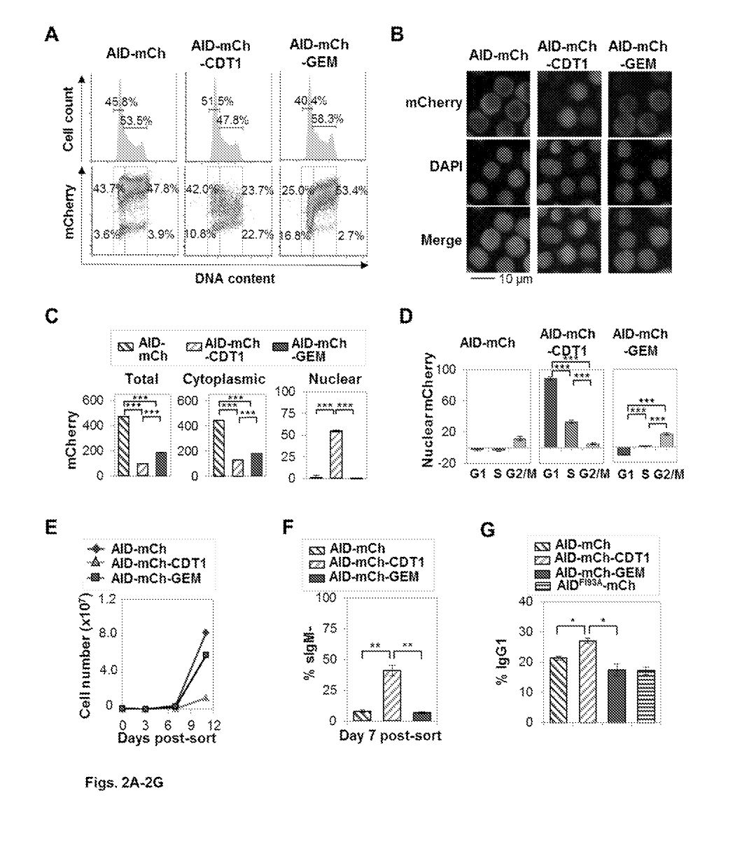

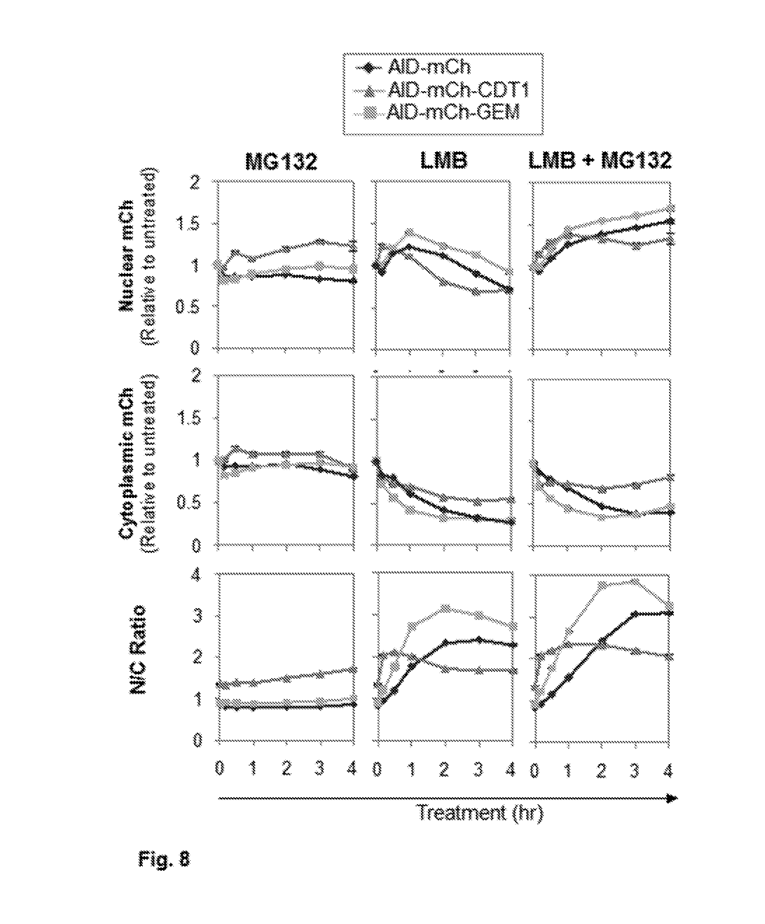

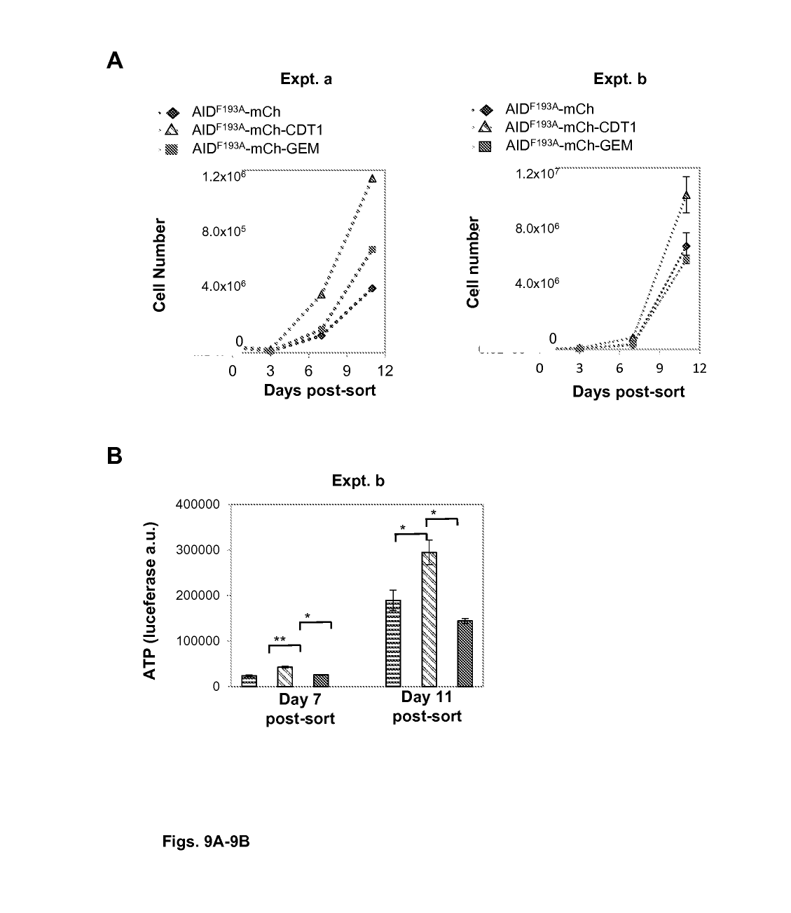

With the goal of restricting the presence of AID-mCherry in the nucleus to G1 or S/G2-M phases, we fused AID-mCherry to tags derived from the CDT1 and GEM cell cycle regulators, which target a fused protein for destruction in the nucleus in S-G2/M phase (CDT1) or G1/early S phase (GEM) (44). Control experiments confirmed that, in Ramos B cells, these tags fused to monomeric Kusabira Orange 2 (mKO2) or monomeric Azami-Green (mAG) promoted nuclear localization and conferred the predicted cell cycle regulation: signals from mKO2-CDT1 or mAG-GEM were restricted to G1 phase or late G1/S-G2/M phase, respectively (FIG. 7). Expression of AID-mCherry-CDT1 or AID-mCherry-GEM did not disrupt the cell cycle profile of Ramos B cells (FIG. 2A). However, regulation directed toward AID seemed to override some predicted effects of each tag. Flow cytometry showed that restriction of the AID-mCherry-CDT1 signal to G1 phase was incomplete (FIG. 2A), in contrast to that of mKO2-CDT1 (FIG. 7A) or of AID-mCherry-GEM (FIG. 2A). Immunofluorescence microscopy identified no nuclear signal among cells expressing AID-mCherry-GEM (FIG. 2B), in contrast to the strong nuclear signal among some (but not all) cells expressing AID-mCherry-CDT1 (FIG. 2B) or mAG-GEM (FIG. 7B). Nonetheless both the CDT1 and GEM tags did target the fusion protein for nuclear degradation during a portion of cell cycle, as predicted, as HCS analysis showed that total and cytoplasmic mCherry signals were significantly lower in AID-mCherry-CDT1 and AID-mCherry-GEM relative to AID-mCherry transductant populations (p=0; FIG. 2C, Table 3). Moreover, active nuclear export was confirmed by showing that treatment with LMB or LMB+MG132 caused a comparable increase in nuclear signal (relative to untreated cells) in AID-mCherry, AID-mCherry-CDT1 and AID-mCherry-GEM transductants (FIG. 8).

TABLE 3
Subcellular Distribution of AID Determined by HCS Microscopy.

| | AID-mCh | | AID-mCh-CDT1 | | AID-mCh-GEM | |
|---|---|---|---|---|---|---|
| | N | mean | N | mean | N | mean |
| Total mCherry | 14666 | 444.2 | 6854 | 130.9 | 17309 | 182.9 |
| Cytoplasmic mCherry | 14666 | 473.6 | 6854 | 95.8 | 17309 | 187.5 |
| Nuclear mCherry | 14666 | 0.9 | 6854 | 54.6 | 17309 | −0.5 |

| | AID-mCh vs. | AID-mCh vs. | AID-mCh-CDT1 vs. |
|---|---|---|---|
| | AID-mCh-CDT1 | AID-mCh-GEM | AID-mCh-GEM |
| | p-value | p-value | p-value |
| Total mCherry | 0 | 0 | 0 |
| Cytoplasmic mCherry | 0 | 0 | 0 |
| Nuclear mCherry | 0 | 0.06 | 0 |

| | | | AID^{F193A}-mCh- | | | |
|---|---|---|---|---|---|---|
| | AID^{F193A}-mCh | | CDT1 | | AID^{F193A}-mCh-GEM | |
| | N | mean | N | mean | N | mean |
| Total mCherry | 13485 | 111.4 | 11790 | 76.5 | 9223 | 83.4 |
| Cytoplasmic mCherry | 13485 | 62.9 | 11790 | 41.6 | 9223 | 44.7 |
| Nuclear mCherry | 13485 | 67.5 | 11790 | 44.8 | 9223 | 47.9 |

| | AID^{F193A}-mCh vs. | | AID^{F193A}-mCh- |
|---|---|---|---|
| | AID^{F193A}-mCh- | AID^{F193A}-mCh vs. | CDT1 vs. AID^{F193A}- |
| | CDT1 | AID^{F193A}-mCh-GEM | mCh-GEM |
| | p-value | p-value | p-value |
| Total mCherry | 0 | 5.09E−260 | 4.0E−19 |
| Cytoplasmic mCherry | 0 | 1.52E−254 | 6.6E−17 |
| Nuclear mCherry | 5.35E−110 | 0 | 2.3E−03 |

The number of cells (N) and the mean total, cytoplasmic, and nuclear mCherry signals are tabulated for Ramos AID-mCherry, AID-mCherry-CDT1, AID-mCherry-GEM, AID^{F193A}-mCherry, AID^{F193A}-mCherry-CDT1 and AID^{F193A}-mCherry-GEM transductants. Nuclear signals as determined by HCS were corrected for cytoplasmic baseline (see Materials and Methods). Statistical tests were performed using two-tailed, unpaired Student's t-test, assuming unequal variances for comparisons among transductant populations.

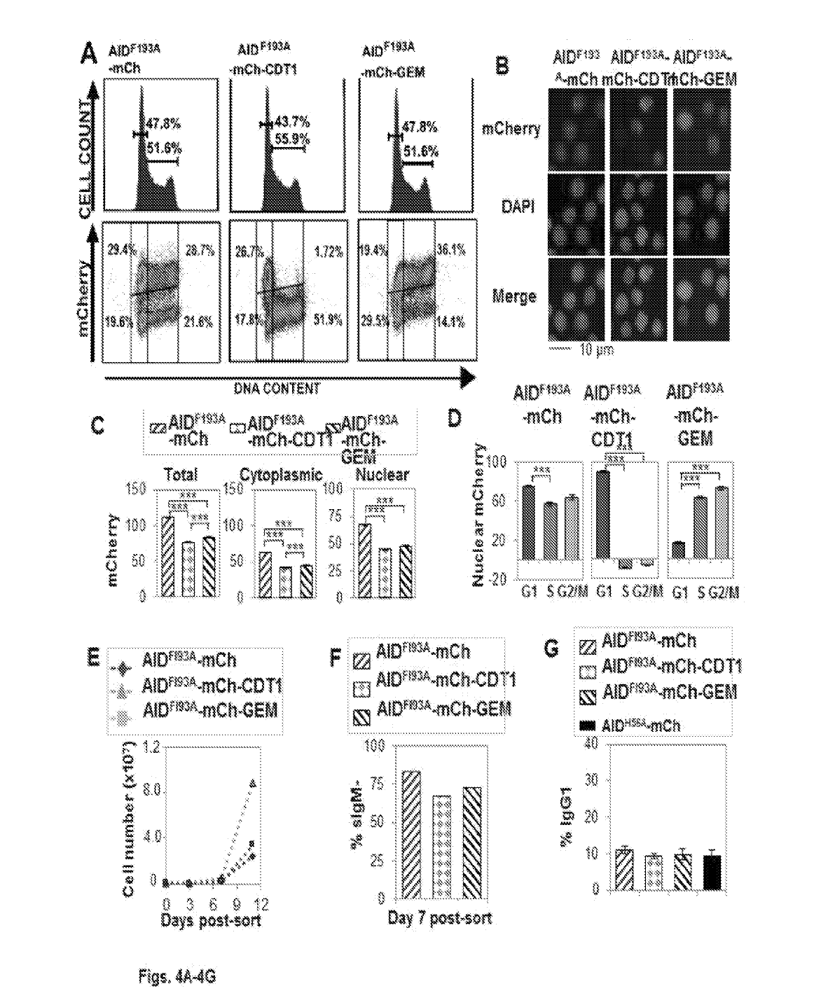

The nuclear localization of the AID-mCherry-CDT1 derivative could reflect more rapid nuclear import. However, while the nuclear signal and the ratio of nuclear to cytoplasmic signal (N/C) peaked more quickly in AID-mCherry-CDT1 than in AID-mCherry or AID-mCherry-GEM transductants following treatment with LMB (FIG. 8), this modest increase does not fully explain the strong nuclear signal in a significant fraction of AID-mCherry-CDT1 transductants. In addition, HCS analysis showed that while AID-mCherry-CDT1 nuclear signal was greatest in G1 phase cells, it was also evident in S phase cells. This suggested that AID-mCherry-CDT1 exported from the nucleus in G1 phase may re-enter in S phase to create a signal before it is targeted for proteolysis by the CDT1 tag. This possibility is addressed experimentally below (FIG. 4).

HCS analysis also showed that AID-mCherry and AID-mCherry-GEM signals were exclusively cytoplasmic, independent of cell cycle (FIG. 2D, Table 4). Combined with the evidence that AID is degraded more rapidly in S and G2-M phases than in G1 phase (FIG. 1), the absence of nuclear signal in AID-mCherry-GEM transductants suggests that mechanisms targeted to the GEM tag promote its nuclear proteolysis in G1 phase, while mechanisms targeted to AID promote its proteolysis in other stages of cell cycle.

TABLE 4
Cell Cycle Cependence of Subcellular Localization of AID.

| | | | | | | | | G1 vs. | S vs. |
|---|---|---|---|---|---|---|---|---|---|
| | G1 | | S | | G2/M | | G1 vs. S | G2/M | G2/M |
| | N | mean | N | mean | N | mean | p-value | p-value | p-value |
| | AID-mCh | | | | | | | | |
| Total mCherry | 5282 | 456.9 | 2730 | 429.7 | 1269 | 495.7 | 2.33E−05 | 1.27E−05 | 6.88E−12 |
| Cytoplasmic mCherry | 5282 | 488.5 | 2730 | 461.8 | 1269 | 525.5 | 0.0002 | 0.0001 | 1.06E−09 |
| Nuclear mCherry | 5282 | −2.5 | 2730 | −3.3 | 1269 | 11.2 | 0.6984 | 5.90E−06 | 5.43E−06 |
| | AID-mCh-CDT1 | | | | | | | | |
| Total mCherry | 1785 | 164.8 | 1285 | 106.0 | 899 | 75.7 | 6.27E−53 | 3.43E−142 | 1.52E−21 |
| Cytoplasmic mCherry | 1785 | 113.4 | 1285 | 80.4 | 899 | 64.8 | 1.54E−33 | 3.87E−69 | 1.05E−08 |
| Nuclear mCherry | 1785 | 88.8 | 1285 | 33.4 | 899 | 5.2 | 3.43E−80 | 2.49E−192 | 3.31E−33 |
| | AID-mCh-GEM | | | | | | | | |
| Total mCherry | 5680 | 95.9 | 3305 | 227.1 | 2011 | 292.9 | 0 | 1.18E−299 | 6.96E−38 |
| Cytoplasmic mCherry | 5680 | 98.2 | 3305 | 235.2 | 2011 | 297.7 | 0 | 5.21E−286 | 1.43E−31 |
| Nuclear mCherry | 5680 | −10.7 | 3305 | 0.9 | 2011 | 16.8 | 1.23E−65 | 6.37E−71 | 3.61E−23 |
| | AID^{F193A}-mCh | | | | | | | | |
| Total mCherry | 5047 | 117.5 | 2281 | 103.2 | 1396 | 109.7 | 1.08E−15 | 5.48E−04 | 8.65E−03 |
| Cytoplasmic mCherry | 5047 | 66.4 | 2281 | 58.6 | 1396 | 60.2 | 3.22E−09 | 4.89E−05 | 3.65E−01 |
| Nuclear mCherry | 5047 | 75.4 | 2281 | 56.8 | 1396 | 63.6 | 2.24E−23 | 5.55E−07 | 7.67E−03 |
| | AID^{F193A}-mCh-CDT1 | | | | | | | | |
| Total mCherry | 5192 | 110.6 | 1649 | 35.9 | 951 | 38.3 | 0 | 0 | 7.58E−06 |
| Cytoplasmic mCherry | 5192 | 50.2 | 1649 | 31.7 | 951 | 32.1 | 3.56E−224 | 5.72E−261 | 4.39E−01 |
| Nuclear mCherry | 5192 | 90.1 | 1649 | −9.9 | 951 | −6.5 | 0 | 0 | 3.45E−06 |
| | AID^{F193A}-mCh-GEM | | | | | | | | |
| Total mCherry | 3049 | 60.0 | 1689 | 94.6 | 1222 | 102.6 | 2.43E−127 | 9.42E−117 | 4.82E−05 |
| Cytoplasmic mCherry | 3049 | 41.5 | 1689 | 45.0 | 1222 | 46.6 | 3.26E−06 | 4.18E−11 | 6.13E−02 |
| Nuclear mCherry | 3049 | 16.6 | 1689 | 63.9 | 1222 | 73.5 | 4.47E−142 | 8.24E−120 | 1.73E−04 |

The number of cells (N) and the mean total, cytoplasmic, and nuclear mCherry signals are tabulated for G1, S and G2/M cells in Ramos AID-mCherry, AID-mCherry-CDT1 and AID-mCherry-GEM, AID^{F193A}-mCherry, AID^{F193A}-mCherry-CDT1, AID^{F193A}-mCherry-GEM transductant populations. Nuclear signals as determined by HCS were corrected for cytoplasmic baseline (see Materials and Methods). Statistical tests were performed using two-tailed, unpaired Student's t-test, assuming unequal variances for comparisons among G1, S and G2/M phase cells in transductant populations.

AID-mCherry-CDT1 Reduced Viablity and Accelerated Ig Gene Diversification.

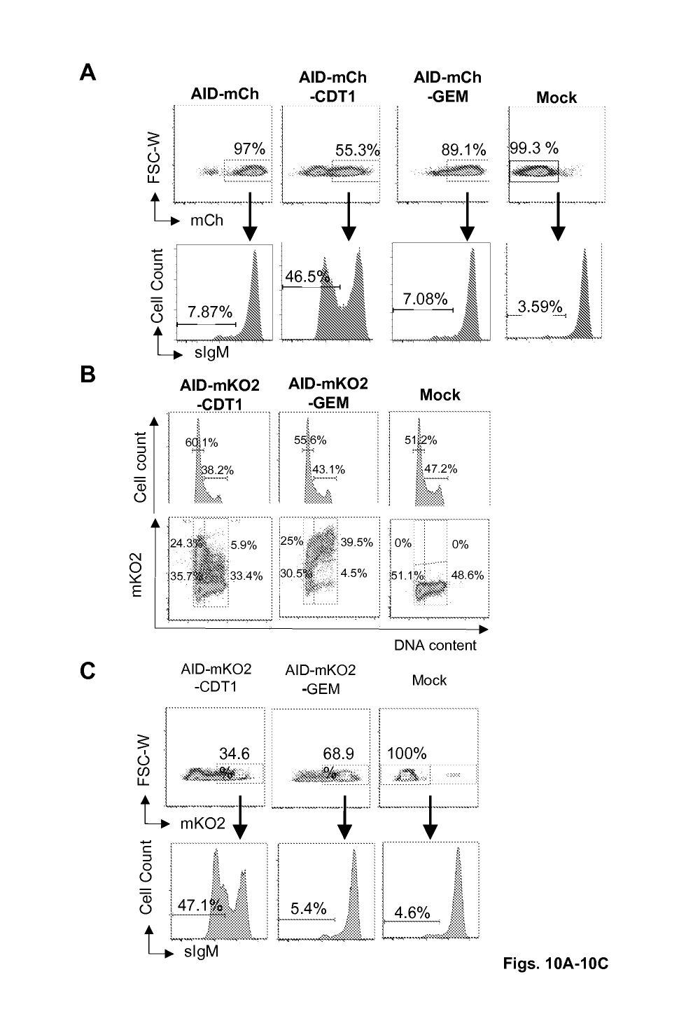

The distinctive spatiotemporal regulation of AID-mCherry, AID-mCherry-CDT1 and AID-mCherry-GEM allowed us to analyze the physiological consequences of nuclear AID at different stages of cell cycle. Strikingly, AID-mCherry-CDT1 transductants exhibited diminished cell viability relative to AID-mCherry or AID-mCherry-GEM transductants (FIG. 2E; FIG. 9). This suggested that nuclear AID can compromise fitness; and we show below (FIG. 4) that the effect on fitness is cell cycle dependent.

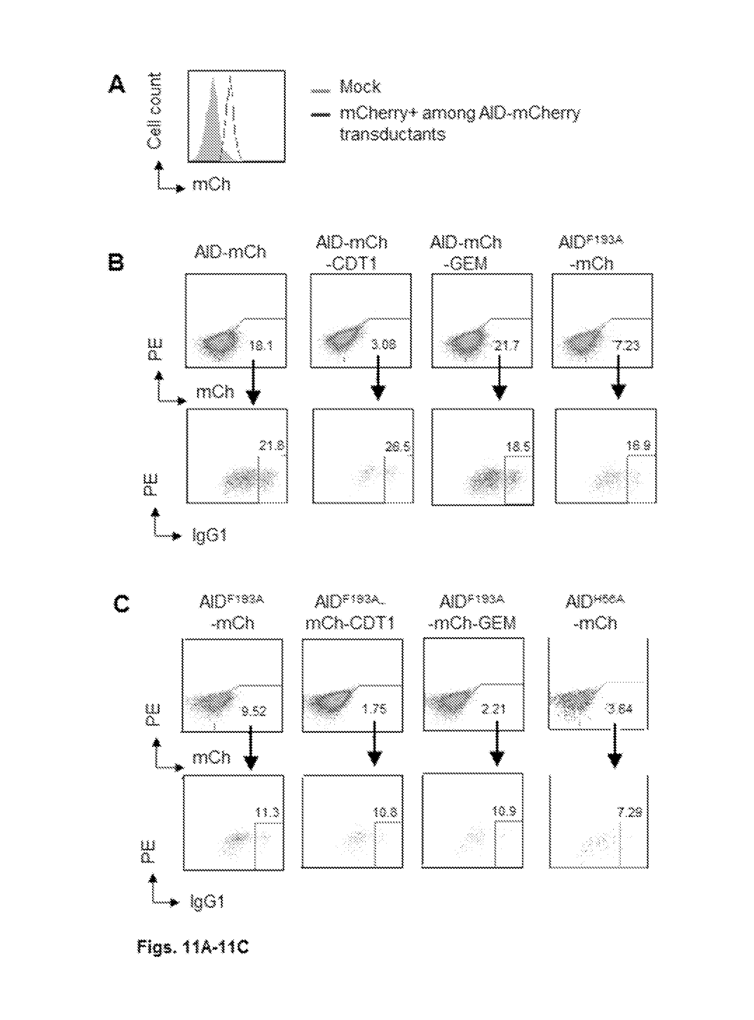

sIgM loss frequency was 7.9% in AID-mCherry transductants, 41.1% (p=0.003) in AID-mCherry-CDT1 transductants, and 6.5% in AID-mCherry-GEM transductants (FIG. 2F; FIG. 10A). Similar results were obtained in assays of Ramos AID-mKO2-CDT1 and AID-mKO2-GEM transductants, which carry an mKO2 fluorescent tag which is degraded more rapidly than the mCherry tag (FIG. 10B, 10C). Thus, the CDT1 tag accelerated AID-initiated SHM in Ramos B cells.

We assayed the effects of the tagged AID derivatives in a more physiological context by transducing primary murine B cells with AID-mCherry, AID-mCherry-CDT1 or AID-mCherry-GEM, and culturing in vitro with IL-4 and anti-CD40 to stimulate CSR. The mCherry signal in transduced primary B cells was too low for HCS analysis (FIG. 11A). Nonetheless, expression of the tagged derivatives had consequences parallel to those observed in Ramos B cells, as among AID-mCherry-CDT1 transductants, a significantly greater average fraction of cells switched to IgG1+(27%) than among AID-mCherry (21%; p=0.006) or AID-mCherry-GEM (18%; p=0.026) transductants (FIG. 2G; FIG. 11B). Thus, AID-mCherry-CDT1 expression accelerated both SHM in the Ramos B cell line and CSR in primary B cells.

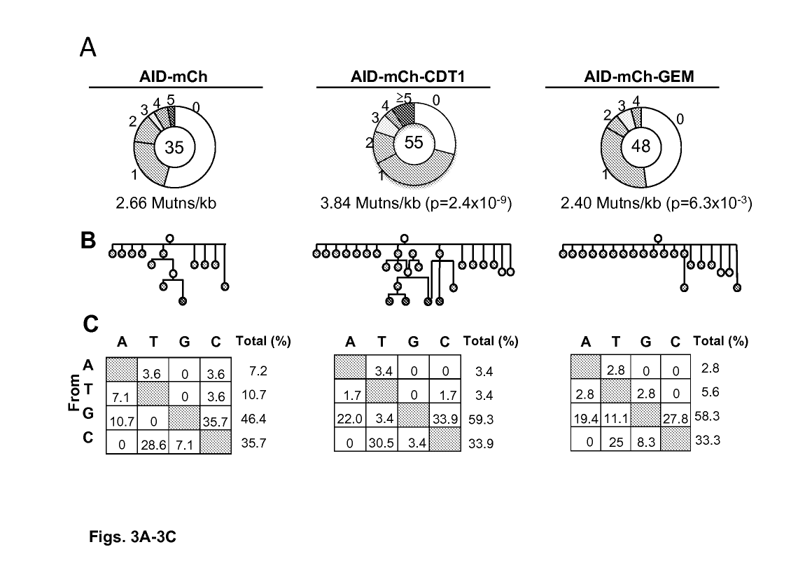

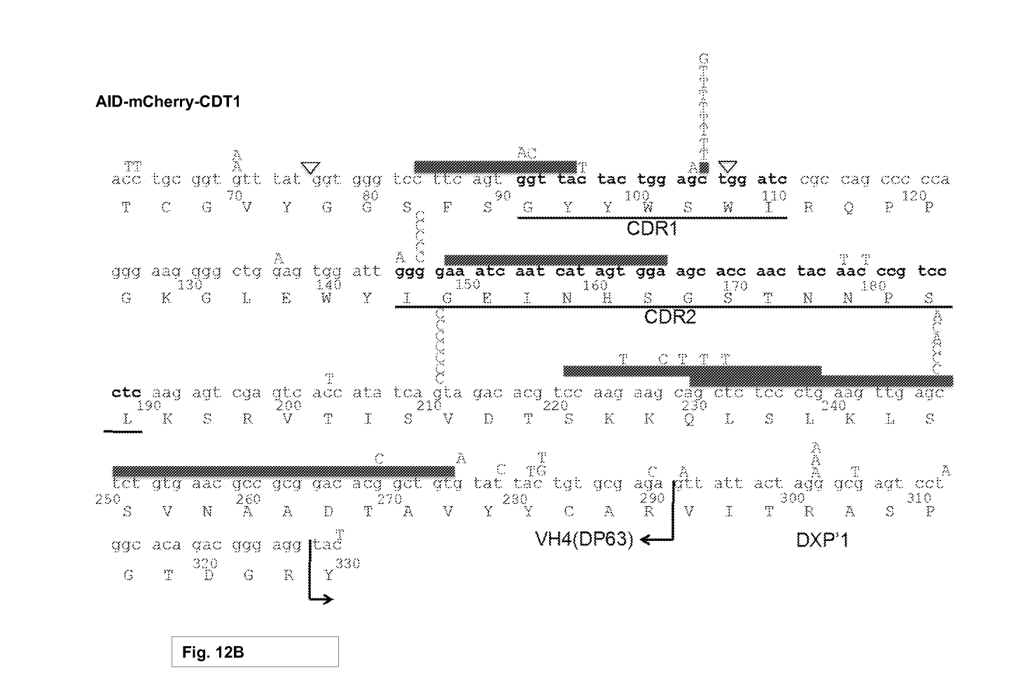

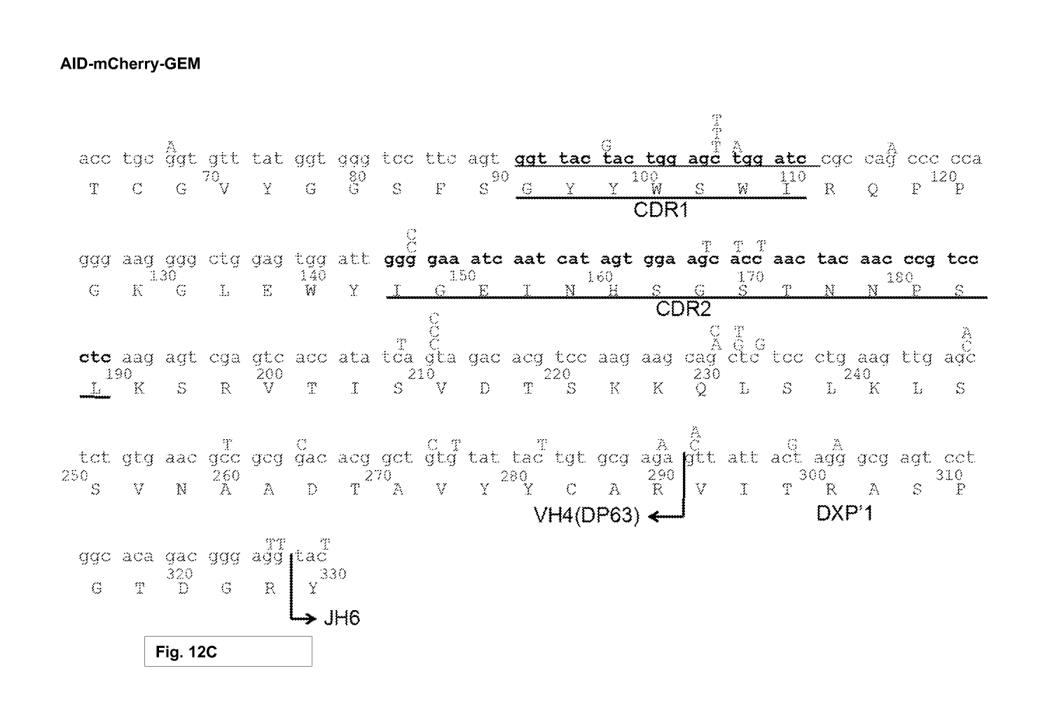

We sequenced IgV_{H} regions amplified from single cells (FIG. 12) to determine mutation frequencies and spectra. AID-mCherry-CDT1 transductants accumulated more mutations and more mutations per V region than AID-mCherry transductants (p=2.4×10¹⁹; FIG. 3A). Point mutations at G or C accounted for over 80% of mutations in all transductants, accompanied by a few deletions and insertions (FIG. 13), similar to other analyses of SHM in Ramos B cells and derivatives expressing AID-GFP (45-47). Accelerated SHM was further evident in diagrams of mutant lineages (FIG. 3B). There were fewer mutations at A or T in AID-mCherry-CDT1 and AID-mCherry-GEM transductants relative to AID-mCherry transductants (6.8%, 8.4% and 17.9%, respectively; FIG. 3C). An especially high fraction of transversion mutations from G to T were evident in AID-mCherry-GEM relative to AID-mCherry or AID-mCherry-CDT1 transductants (11.1%, 0% and 3.4% respectively; FIG. 3C).

Elevated Nuclear AID is Tolerated in G1 Phase but not in S-G2/M Phase Cells.

The presence of a nuclear AID-mCherry-CDT1 signal in both G1 and S phase cells (FIG. 2D) suggested that AID-mCherry-CDT1 that is exported from the nucleus in G1 phase can re-enter in S phase, generating a nuclear signal until it is targeted for proteolysis by the CDT1 tag. To test this, we analyzed spatiotemporal localization of derivatives carrying the well-characterized AID^{F193A} mutation, which prevents nuclear export, reduces protein levels and accelerates SHM (36). Flow cytometry showed that expression of AID^{F193A}-mCherry, AID^{F193A}-mCherry-CDT1 or AID^{F193A}-mCherry-GEM did not disrupt the cell cycle profile in Ramos B cells (FIG. 4A). Fluorescence microscopy identified clear nuclear signals in each transductant population, consistent with inhibition of nuclear export (FIG. 4B). In the AID^{F193A}-mCherry-CDT1 transductant population, essentially no S-G2/M phase cells exhibited mCherry signal, in contrast to AID-mCherry-CDT1 transductants (cf. FIGS. 2A and 4A; 2D and 4D). This establishes that nuclear export and re-entry is the source of the AID-mCherry-CDT1 nuclear signal.

HCS analysis showed that total and cytoplasmic mCherry signals were significantly lower in AID^{F193A}-mCherry-CDT1 and AID^{F193A}-mCherry-GEM transductants than in AID^{F193A}-mCherry transductants, as predicted for tags that target the protein for nuclear degradation during a portion of cell cycle (FIG. 4C). Comparison of AID-mCherry vs. AID^{F193A}-mCherry and AID-mCherry-GEM vs. AID^{F193}-mCherry-GEM transductants showed that the F193A mutation reduced total and cytoplasmic signals several-fold or more, and greatly increased nuclear signals; while signals were reduced to a lesser extent in AID-mCherry-CDT1 relative to AID^{F193}-mCherry-CDT1 transductants (cf. FIGS. 2C and 4C).

HCS documented persistent nuclear localization of AID^{F193A}-mCherry and AID^{F193A}-mCherry-GEM in all phases of cell cycle, while nuclear localization of AID^{F193A}-mCherry-CDT1 occurred exclusively in G1 phase (FIG. 4D). AID^{F193A}-mCherry and AID^{F193A}-mCherry-GEM transductants exhibited diminished cell viability, but AID^{F193A}-mCherry-CDT1 transductants proliferated robustly (FIG. 4E). We conclude that cells tolerate high levels of nuclear AID provided that it is restricted to G1 phase, but do not tolerate nuclear AID at other stages of cell cycle.

The Ramos AID^{F193A}-mCherry, AID^{F193A}-mCherry-CDT1 and AID^{F193A}-mCherry-GEM transductants all exhibited greatly elevated sIgM loss rates (FIG. 4F), as previously documented for AID^{F193A} mutants (36). However, CSR to IgG1 was not accelerated in primary B cells expressing AID derivatives bearing the F193A mutation (FIG. 4G; FIG. 11C), as expected because CSR requires an intact AID C-terminal region (36, 37).

AID^{F193A}-mCherry-CDT1 was distinguished by its ability to accelerate SHM without vastly compromising cell viability. This will make AID^{F193A}-mCherry-CDT1 a useful tool for accelerating mutagenesis in platforms designed to optimize evolution of antibodies and other targets.

### Discussion

We have shown that cell cycle regulates AID nuclear stability and the cellular response to AID. The role of cell cycle regulation of AID-initiated mutagenesis has previously been elusive. Although total AID levels had been found to remain constant during cell cycle (31, 36), evidence that AID-initiated DNA damage occurred in G1 phase (39-42) had suggested that temporal regulation might be important. We have distinguished nuclear from total AID levels, to demonstrate that AID is degraded in the nucleus more slowly in G1 than S-G2/M phases, and that G1 phase nuclear AID accelerates SHM and CSR, without compromising cell viability. Thus, G1 phase is the sweet spot for AID-initiated mutagenesis.

The unanticipated resilience of G1 phase cells to AID-initiated damage was especially evident in the contrast between the high viability of AID^{F193A}-mCherry-CDT1 transductants, in which AID is in the nucleus only in G1 phase, and the poor viability of AID^{F193A}-mCherry and AID^{F193A}-mCherry-GEM transductants, in which AID is in the nucleus outside G1 phase (FIG. 4E). Restriction of nuclear AID to G1 phase will limit the ability of AID to initiate genomic instability, by preventing access to DNA when it becomes transiently single-stranded during replication in S phase. Nonetheless, G1 phase AID will be able to access single-stranded regions within transcribed genes. Deaminated DNA (particularly within transcribed regions) may be repaired more efficiently in G1 phase than in other phases of cell cycle, reversing this initial damage caused by AID.

The GEM tag was predicted to restrict nuclear protein to S-G2/M phase, but there was no nuclear AID-mCherry-GEM signal in any stage of cell cycle. Nuclear AID is degraded more slowly in G1 than S or G2/M phase (FIG. 1). Our results argue that the AID-mCherry-GEM fusion protein was eliminated from the nucleus in G1 phase by degradation targeted to the GEM tag, and that it was eliminated from the nucleus in S phase by degradation targeted to AID itself. AID has eight lysine targets for ubiquitination (31), and differential ubiquitination may be one source of temporal regulation.

AID^{F193A}-mCherry-GEM accumulated in the nucleus during S-G2/M phase, while AID-mCherry-GEM did not (compare FIGS. 2D and 4D). This suggests that nuclear export directed to AID overrides nuclear import specified by the two NLS's in the GEM tag. We note that cell cycle may also differentially regulate nuclear export of AID in G1 and S-G2/M phases, a possibility that can be addressed in future experiments.

The CDT1 tag destabilizes nuclear protein outside G1 phase (44) and would not be predicted to increase nuclear levels at any stage of cell cycle. Nonetheless, AID-mCherry-CDT1 nuclear signal exceeded that of AID-mCherry (FIG. 3C, D). This somewhat paradoxical result could be explained if the CDT1 tag enabled more efficient nuclear import. Consistent with this, treatment with LMB or LMB+MG132 did cause a more rapid increase in nuclear signal in AID-mCherry-CDT1 than AID-mCherry transductants (FIG. 8), but the modest difference observed is unlikely to provide a complete explanation. Alternatively, we speculate that AID may be regulated by feedback loops that determine nuclear levels in G1 phase based on the level in another compartment or stage of cell cycle. A cell that has not carried out Ig gene diversification in one cell cycle may be favored to do so in the next, in which case low levels of AID in G2/M phase may lead to elevated nuclear levels in the next G1 phase, as was evident in the AID-mCherry-CDT1 transductants (FIG. 2D).

The CDT1 and GEM tags somewhat altered the spectrum of SHM. A reduced frequency of mutations at A and T was evident in AID-mCherry-CDT1 (6.8%) and AID-mCherry-GEM (8.4%) relative to AID-mCherry transductants (17.9%; FIG. 3). An especially high fraction of transversion mutations from G to T was evident in AID-mCherry-GEM transductants (11.1%) relative to AID-mCherry (0%) or AID-mCherry-CDT1 transductants (3.4%; FIG. 4D). This class of mutations can be generated by activity of Rev1 (48) or Polη (49). The reduced level of mutations at A and T argues against a predominant role for Polη, which is especially active at mutating at A and T (50). Instead Rev1 may be responsible for the G to T transversions in AID-mCherry-GEM transductants. This suggests that Rev1 may function late in cell cycle. Consistent with this, Rev1 has been shown to repair UV damage at gaps that persist into G2 phase (51).

The use of CDT1 and GEM tags to destabilize nuclear protein outside specific windows of cell cycle (44) proved unexpected insights into regulation of AID and the response to AID-initiated DNA damage. These tags can be readily applied to study repair in other contexts, and they should also prove useful for optimizing the nucleases (CRISPR/Cas9, TALENs, etc.) that target nicks and double-strand breaks for genome engineering and gene correction applications. The utility of these tags is especially evident in the AID^{F193A}-mCherry-CDT1 derivative. AID^{F193A}-mCherry-CDT1 expression greatly accelerates hypermutation, but without the negative impact on cell proliferation associated with other AID derivatives that increase the frequency of SHM but compromise cell viability, including AID mutants selected for increased deamination activity (24); NES mutants (36, 37); and the naturally occurring human AIDΔE5 dominant negative mutant, which exhibits increased hypermutation activity coupled with diminished cell viability (38). AID^{F193A}-mCherry-CDT1 should prove to be useful for defining the mechanisms that protect the genome from AID-initiated DNA damage in G1 phase, and in very practical applications directed toward evolving or optimizing antibodies and other proteins.

### Materials and Methods

Expression Constructs.

The pEGFP-N3 construct for expression of AID-GFP was a gift from Dr. Javier Di Noia (Department of Microbiology and Immunology, University of Montreal, Montreal, Quebec, Canada). We substituted mCherry for a region of GFP flanked by ApaI and BsrGI restriction sites in the pEGFP-N3 construct to generate an AID-mCherry expression construct, pAID-mCh. Cell cycle reporter constructs p-mKO2-CDT1 CSII and p-mAG-GEM CSII, in a lentiviral vector, were a gift from Dr. Atsushi Miyawaki (Brain Science Institute, RIKEN, Hirosawa, Wako-city, Saitama 351-0198, Japan).

pAID-mCh CSII: We amplified AID-mCherry from pAID-mCh with primers PQL31, 5′-ATATCAATTGAGATCCCAAATGGACAGCC-3′ (SEQ ID NO: 7) and PQL32, 5′-ATATTCTAGATTACTTGTACAGCTCGTCCATGC-3′, (SEQ ID NO: 8) and inserted it between EcoRI and XbaI sites in p-mAG-GEM CSII, thereby replacing mAG-GEM with AID-mCherry.

pAID-mCh-CDT1 and pAID-mCh-GEM: We amplified CDT1 with primers PQL44 5′-TATATGTACAAGGGATATCCATCACACTGGCGGCC-3′ (SEQ ID NO: 9) and PQL45 5′-TATATGTACATCTAGATTAGATGGTGTCCTGGTCC-3′ (SEQ ID NO: 10) from p-mKO2-CDT1 CSII, and GEM with primers PQL44 5′-TATATGTACAAGGGATATCCATCACACTGGCGGCC-3′ (SEQ ID NO: 9) and PQL46 5′-TATATGTACATCTAGATTACAGCGCCTTTCTCCG-3′ (SEQ ID NO: 11) from p-mAG-GEM CSII, and inserted the resulting fragments between BsrGI and XbaI restriction sites of pAID-mCh CSII.

pAID-mKO2-CDT1 and pAID-mKO2-GEM: We amplified mKO2 with primers mKO2 FOR 5′-ATATGGATCCATCGCCACCATGGTGAGTGTG-3′ (SEQ ID NO: 12) and mKO2 REV 5′-ATATGCGGCCGCCAGTGTGATGGATATCCGC-3′ (SEQ ID NO: 13), and inserted the resulting fragment between BamHI and NotI restriction sites in pAID-mCh-CDT1 or pAID-mCh-GEM CSII, respectively.

pAID^{F193A}-mCh-CDT1, pAID^{F193A}-mCh-CDT1 and pAID^{F193A}-mCh-GEM: F193A mutants were generated using QuikChange II XL Site-Directed Mutagenesis Kit (Agilent) with primer set, F193A FOR 5′-CTTACGAGACGCAGCTCGTACTTTGGGAC-3′ (SEQ ID NO: 14) and F193A REV 5′-GTCCCAAAGTACGAGCTGCGTCTCGTAAG-3′(SEQ ID NO: 15).

Cell Culture and Transduction.

The human Burkitt lymphoma cell line, Ramos, was cultured in supplemented RPMI 1640 (Gibco), which contained 10% FBS, 2 mM L-glutamine, penicillin/streptomycin, 1× non-essential amino acids (Gibco), 1 mM sodium pyruvate, and 10 mM HEPES. Lentiviral transductions used 2×10⁵ cells cultured in medium containing 8 μg/ml of polybrene. Following transduction, cells were cultured for 3-4 days and these recent transductants then sorted for mCherry+ to enrich for transduced cells, typically constituting 0.1-10% of the population. Cells were treated with leptomycin B (LMB; LC Laboratories) at 50 ng/ml and MG132 (Z-Leu-Leu-Leu-aldehyde; Sigma-Aldrich) at 50 μM. Viable cells were counted after trypan blue staining. Cell viability was confirmed by CellTiter-Glo® Luminescent Cell Viability Assay (Promega).

Assays of Cell Cycle.

To determine cell cycle distribution, cells were fixed, permeabilized with 0.5% Triton X-100, stained with DAPI (2 μg/ml) and analyzed by FACS.

High Content Screening (HCS) Microscopy and Analysis.

Cells were fixed in 3.7% formaldehyde at a density of 2×10⁶ cells/ml and stained with whole cell stain (HCS CellMask, Invitrogen) and DAPI (0.2 μg/ml). Fixed cells were then washed, resuspended in PBS and spun down in a 96-well μclear microplate (Greiner Bio One) for imaging. Cells were imaged by Thermo Scientific ArrayScan VTI HCS reader, analyzing 3000-6000 cells in each treatment group. Cells with very low or very high mCherry signals were eliminated, gating based on the mock transduction control (low) and eliminating cells with signals more than 5 SD from the mean (high). The HCS Colocalization BioApplication protocol was used to determine nuclear and whole cell boundaries in individual cells as defined by DAPI and HCS CellMask, respectively, thereby defining the cytoplasmic region as the region between nuclear and whole cell boundaries. The average signal in the nuclear and cytoplasmic compartments was determined in individual cells by measuring the total intensity of mCherry signal divided by area within each compartment. The ratio of nuclear to cytoplasmic signal (N/C) was calculated as the ratio of the average signals of nuclear and cytoplasmic mCherry.

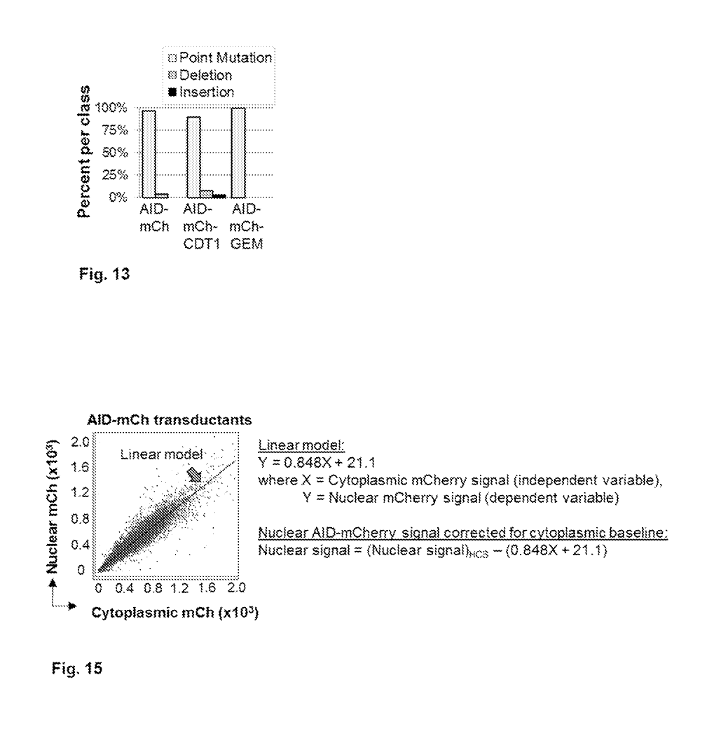

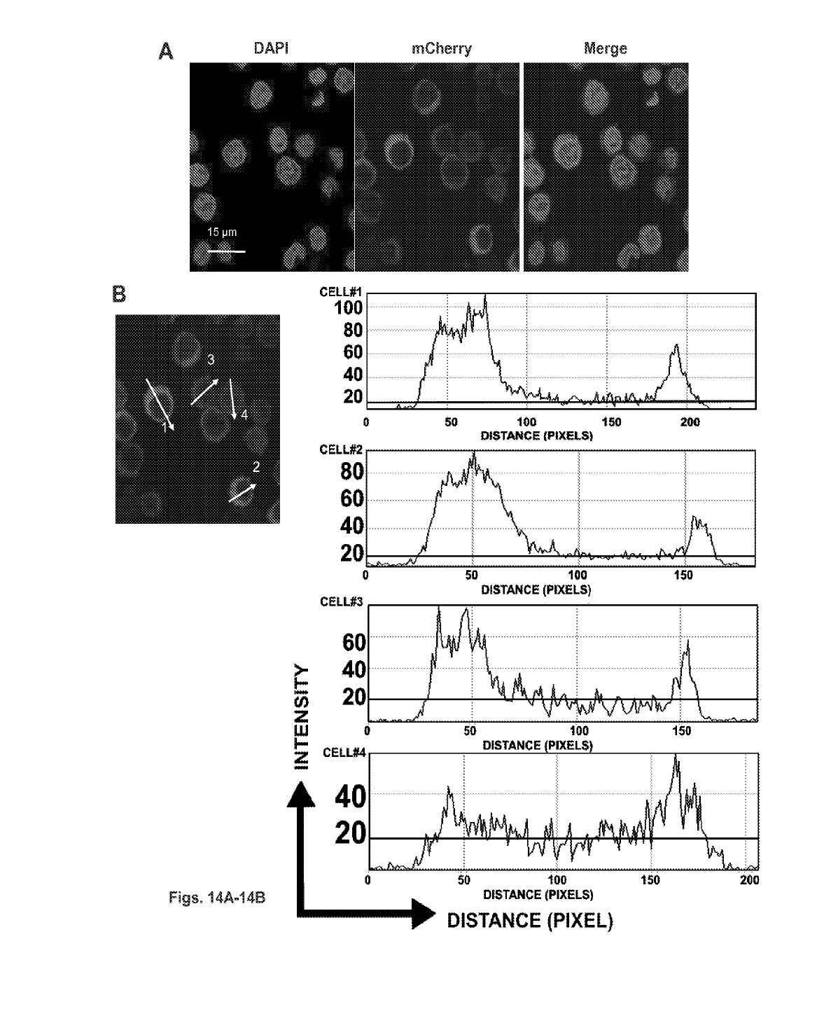

Confocal microscopy showed that AID-mCherry was mostly absent from the nucleus when out-of-focus signal was eliminated, regardless of the level of cytoplasmic signal (FIG. 14). HCS analysis of AID-mCherry transductants showed that nuclear AID-mCherry signals increased linearly with increasing cytoplasmic signals (slope of linear regression=0.848; FIG. 15), consistent with a contribution of cytoplasmic signal from above or below the nucleus to the signal identified as nuclear by HCS. Thus in order to enable accurate comparisons among AID-mCherry, AID-mCherry-CDT1, AID-mCherry-GEM, AID^{F193A}-mCherry, AID^{F193A}-mCherry-CDT1, and AID^{F193A}-mCherry-GEM transductants, the nuclear signal for each cell, as determined by HCS, was corrected by subtraction of the corresponding baseline value, as established by linear regression analysis of nuclear vs. cytoplasmic signals of untreated AID-mCherry transductants (FIG. 15), using the formula: Nuclear signal=(Nuclear signal)_{HCS}−(0.848X+21.1).

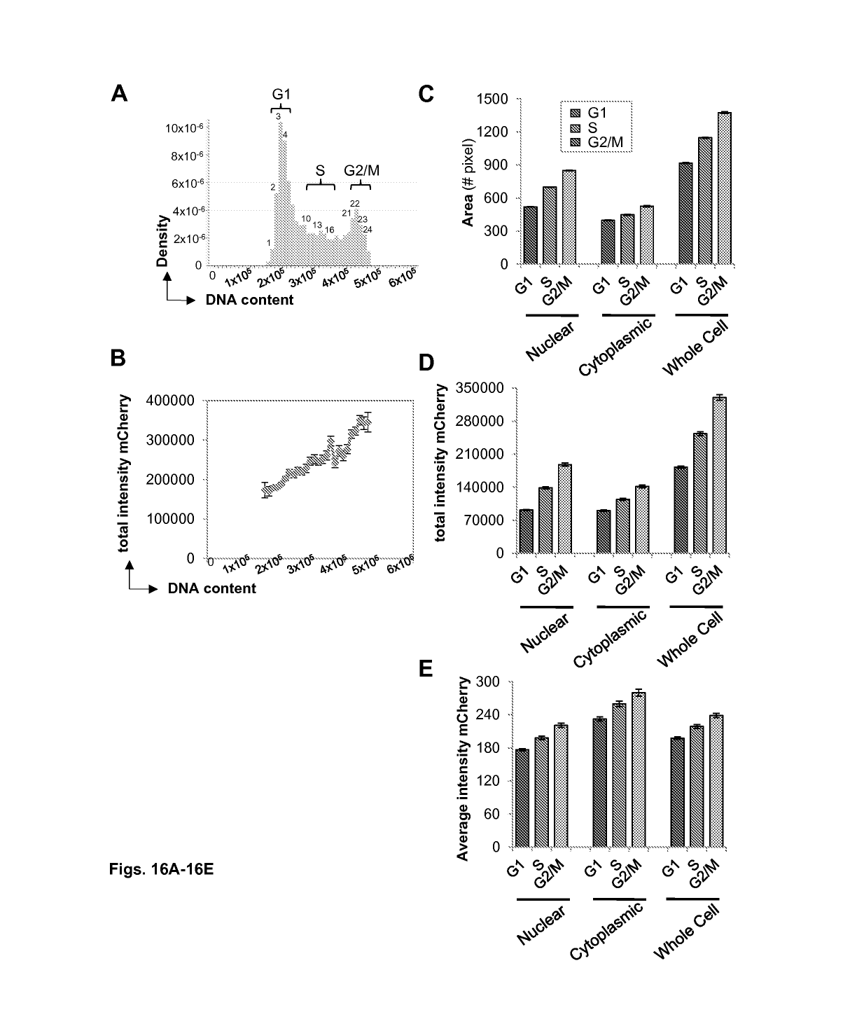

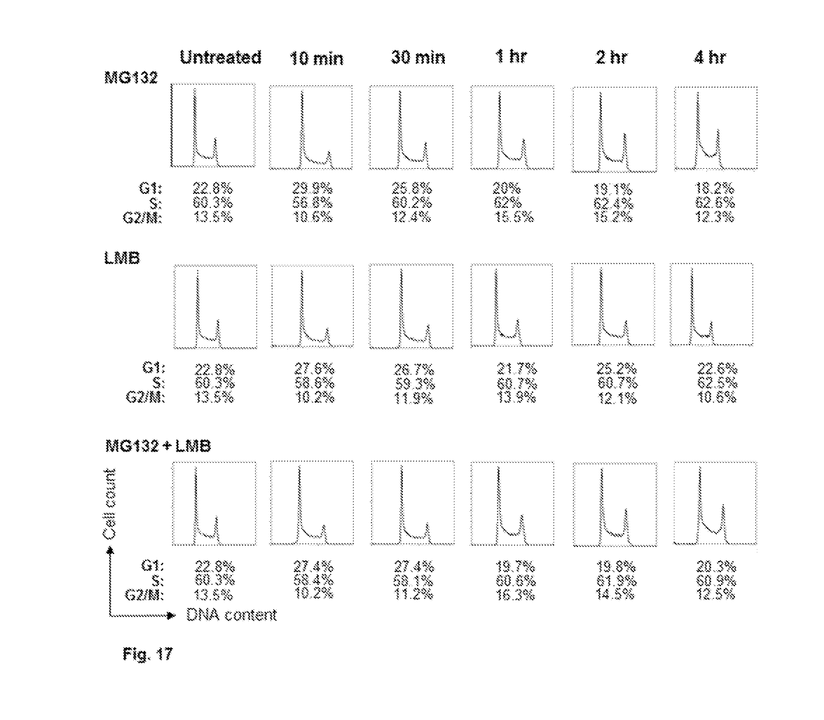

G1, S, and G2/M phase cells were distinguished by ranking DNA content as determined by total DAPI signal, and specific fractions of the population assigned to G1, S and G2/M phases (FIG. 16A). HCS results were expressed in terms of average signal, to ensure independence of cell size, which increases during cell cycle (FIG. 16). Control experiments verified that cell cycle was not perturbed significantly by up to 4 hr of culture with MG132, LMB or MG132+LMB (FIG. 17).

Assays of sIgM Loss Frequency in Ramos B Cell Transductants.

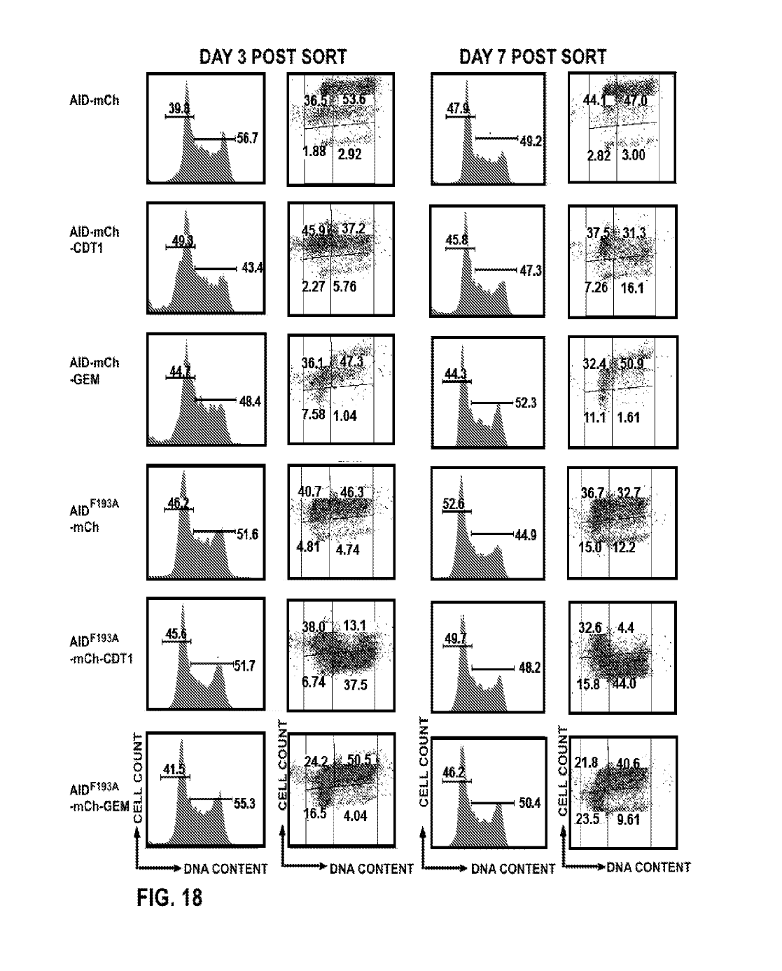

sIgM loss frequency provides a convenient surrogate assay for SHM (45, 46). To determine fractions of sIgM− cells, 2-5×10⁵ cells were fixed in 3.7% formaldehyde and stained with anti-human IgM (1:500, Souther Biotech), and sIgM− variants quantified by FACS as described (47). To establish that selective pressure was not sufficient to affect the frequency of sIgM loss, we assayed loss of mCherry signal posttransduction (FIG. 18). There was modest loss of mCherry expression between days 3 and 7 in the AID-mCherry-CDT1 transductants (decrease from 37.2% to 31.3%), consistent with some selective pressure against AID-mCherry-CDT1 expression, but not sufficient to alter interpretation of the sIgM loss data.

Assay of CSR in Primary Splenic B Cells.

B cells were isolated from spleens of C57BL/6 mice and enriched through a negative selection in AUTOMACs with biotinylated anti-CD43 antibody (BD Pharmigen, Cat #5532269) and streptavidin magnetic microbeads (Miltenyi Biotech, Cat #130-048-102). Purified B cells were transduced for 24 hr in X-vivo medium (Lonza) containing 2 mM L-glutamine, 50 μM β-mercaptoethanol, 5 ng/mL IL-4 (R&D Systems, cat#404-ML-010) and 1 μg/mL anti-CD40 antibody (BioLegend, Cat#102802) in 100 μL total volume in a round bottom 96-well plate, then transferred at 24 hr to supplemented RPMI (see above) containing 5 ng/mL IL-4 and 1 μg/mL anti-CD40 antibody. Cells were cultured for 4-5 days, stained with anti-IgG1 (FITC anti-mouse IgG1; BioLegend, Cat#406605), and surface IgG1 quantified by flow-cytometry.

Single-Cell PCR and Sequencing of V_{H} Regions.

At day 7 post sorting recent transductants for mCherry+ cells, single cells from AID-mCherry, AID-mCherry-CDT1 or AID-mCherry-GEM transductant populations were aliquoted, one cell per well, into 96-well plates containing 20 μl of Pfu reaction buffer (Agilent). Samples were frozen, thawed, and treated with 250 μg/ml proteinase K for 1 hr at 50° C. then 5 min at 95° C., the primers and high-fidelity Pfu Turbo DNA polymerase (Agilent) were added and the rearranged V_{H} region amplified by nested PCR with first round primers, RV_{H}FOR QL 5′-TCCCAGGTGCAGCTACAGCAG-3′ (SEQ ID NO: 16) and JOL48 QL 5′-GTACCTGAGGAGACGGTGACC-3′ (SEQ ID NO: 17) (52); followed by 1:30 dilution and second round amplification with primers 5′-AGGTGCAGCTACAGCAGTG-3′ (SEQ ID NO: 18) and 5′-GCCCCAGACGTCCATACC-3′ (SEQ ID NO: 19). Predicted sizes of PCR products were confirmed by gel electrophoresis and fragments purified and sequenced.

Cell Culture and Transduction.

Ramos B cells were transduced in medium containing polybrene, cultured for 3-4 days, then sorted for mCherry+ to enrich for transduced cells, typically constituting 0.1-10% of the population. Primary murine B cells were transduced in supplemented X-vivo medium, then cultured 4-5 days with IL-4 and anti-CD40, and the fraction of IgG1+ cells quantified.

High Content Screening (HCS) Microscopy.

Cells were fixed and stained with whole cell stain (HCS CellMask, Invitrogen) and DAPI, washed, and imaged by Thermo Scientific ArrayScan VTI HCS reader, analyzing 3000-6000 cells in each treatment group. To enable accurate comparisons among different transductant populations, nuclear signal for each cell was corrected by subtraction of the corresponding baseline value, as established by linear regression analysis. HCS results were expressed in terms of average signal, to ensure independence of cell size.

### REFERENCES

1. Muramatsu M, et al. (2000) Cell 102:553-563.
2. Revy P, et al. (2000) Cell 102:565-575.
3. Maizels N (2005) Annu Rev Genet 39:23-46.
4. Di Noia J M & Neuberger M S (2007) Annu Rev Biochem 76:1-22.
5. Lee-Theilen M & Chaudhuri J (2010) Nat Immunol 11:107-109.
6. Storck S, et al. (2011) Curr Opin Immunol 23:337-344.
7. Hasler J, et al. (2012) Semin Immunol 24:273-280.
8. Gazumyan A, et al. (2012) Adv Cancer Res 113:167-190.
9. Ramiro A R, et al. (2004) Cell 118:431-438.
10. Pasqualucci L, et al. (2004) Blood 104:3318-3325.
11. Morgan H D, et al. (2004) J Biol Chem 279:52353-52360.
12. Popp C, et al. (2010) Nature 463:1101-1105.
13. Bhutani N, et al. (2010) Nature 463:1042-1047.
14. Munoz D P, et al. (2013) Proc Natl Acad Sci USA 110:E2977-2986.
15. Kumar R, et al. (2013) Nature 500:89-92.
16. Kuraoka M, et al. (2011) Proc Natl Acad Sci USA 108:11560-11565.
17. Meyers G, et al. (2011) Proc Natl Acad Sci USA 108:11554-11559.
18. Durandy A, et al. (2013) Autoimmunity 46:148-156.
19. Zan H & Casali P (2013) Autoimmunity 46:83-101.
20. Liu M, et al. (2008) Nature 451:841-845.
21. Takizawa M, et al. (2008) J Exp Med 205:1949-1957.
22. Robbiani D F, et al. (2009) Mol Cell 36:631-641.
23. Yamane A, et al. (2011) Nat Immunol 12:62-69.
24. Wang M, et al. (2009) Nat Struct Mol Biol 16:769-776.
25. Chaudhuri J, et al. (2003) Nature 422:726-730.
26. Ramiro A R, et al. (2003) Nat Immunol 4:452-456.
27. Bransteitter R, et al. (2003) Proc Natl Acad Sci USA 100:4102-4107.
28. Pham P, et al. (2003) Nature 424:103-107.
29. Dickerson S K, et al. (2003) J Exp Med 197:1291-1296.
30. Nabel C S, et al. (2013) Proc Natl Acad Sci USA 110:14225-14230.
31. Aoufouchi S, et al. (2008) J Exp Med 205:1357-1368.
32. Orthwein A, et al. (2010) J Exp Med 207:2751-2765.
33. McBride K M, et al. (2004) J Exp Med 199:1235-1244.
34. Ito S, et al. (2004) Proc Natl Acad Sci USA 101:1975-1980.
35. Brar S S, et al. (2004) J Biol Chem 279:26395-26401.
36. Geisberger R, et al. (2009) Proc Natl Acad Sci USA 106:6736-6741.
37. Uchimura Y, et al. (2011) J Exp Med 208:2385-2391.
38. Zahn A, et al. (2014) Proc Natl Acad Sci USA 111:E988-997.
39. Ordinario E C, et al. (2009) J Immunol 183:4545-4553.
40. Yabuki M, et al. (2009) J Immunol 182:408-415.
41. Sharbeen G, et al. (2012) J Exp Med 209:965-974.
42. Yamane A, et al. (2013) Cell Rep 3:138-147.
43. Li M M & Emerman M (2011) J Virol 85:8197-8207.
44. Sakaue-Sawano A, et al. (2008) Cell 132:487-498.
45. Sale J E, et al. (2001) Nature 412:921-926.
46. Rada C, et al. (2002) Proc Natl Acad Sci USA 99:7003-7008.
47. Yabuki M, et al. (2005) Nat Immunol 6:730-736.
48. Jansen J G, et al. (2006) J Exp Med 203:319-323.
49. Kano C, et al. (2012) Int Immunol 24:169-174.
50. Masuda K, et al. (2007) J Biol Chem 282:17387-17394.
51. Diamant N, et al. (2012) Nucleic Acids Res 40:170-180.
52. Sale J E & Neuberger M S (1998) Immunity 9:859-869.

### Example 2

### Modulation and Optimization of Chimeric Antigen Receptor T Cells

This example illustrates an embodiment of the invention that implements the principles described above for use with B cells to T cells. More specifically, one can use the invention described herein to modulate and optimize chimeric antigen receptor (CAR) T cells for use in therapeutic treatments. One can modulate and improve the affinity or specificity of a CAR T cell by transfecting a host T cell with a fusion construct of the invention. The fusion construct would couple a fragment of a protein targeted for nuclear destruction during a relevant portion of the cell cycle (e.g., CDT1 for destruction upon entry into S phase; GEM for G1 phase destruction) with AID modified to promote accumulation of AID in the nucleus. This construct stimulates diversification of the target gene to be optimized for immunotherapeutic use.

### Example 3

### Modulation of Nuclear Protein Activity

This example illustrates an embodiment of the invention, whereby cell cycle tags derived from CDT1 or GEM (or other proteins involved in cell cycle control) can confer cell cycle restriction to enzymes that function in the nucleus. This modulation of nuclear protein activity can be of use, for example, in genome engineering. The nuclease activities of enzymes used to target DNA and the pathways of downstream repair can reflect the stage of cell cycle in which the DSB or nick occurs. For example, the frequency of a desired outcome (e.g. homology-directed repair) would be higher if DNA is cleaved in G1 phase, by an enzyme bearing a CDT1 tag; or the frequency of an undesired outcome (mutagenic end-joining) would be lower if DNA is cleaved in S phase, by an enzyme bearing a GEM tag.

Two enzymes widely used for genome engineering are CRISPR/Cas9, which creates targeted double-strand breaks (DSBs); and the CRISPR/Cas9D10A nickase, which creates targeted single-strand breaks (nicks). This can be implemented by using standard cloning approaches to generate constructs that express Cas9-CDT1 and Cas9-GEM or Cas9D10A-CDT1 and Cas9D10A-GEM fusion proteins. These fusion proteins will be expressed upon transfection of cultured cells, and predicted cell cycle regulation confirmed by flow cytometry. Frequencies of homology-directed repair, targeted deletions and mutagenic end-joining can be measured, using standard published approaches (e.g. Davis and Maizels, PNAS, 111(10):E924-32, 2014). Comparison of these frequencies can be used to identify optimum stages of cell cycle (and corresponding fusion proteins) for genome engineering.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A nucleic acid construct comprising:
(a) a first nucleotide sequence that expresses an activation-induced cytosine deaminase (AID) polypeptide, wherein the AID polypeptide is modified to prevent nuclear export; and
(b) a second nucleotide sequence that expresses chromatin licensing and DNA replication factor 1 (CDT1) or another polypeptide targeted for cell cycle-dependent nuclear destruction, wherein the second nucleotide sequence is operably linked to the first nucleotide sequence.

2. The nucleic acid construct of claim 1, wherein the AID is AID^{F193A}, AID^{F193E}, AID^{F193H}, AID^{L196A}, AID^{F198A}, AID^{L198S}, AID^{193X} or AID^{196X}.

3. The nucleic acid construct of claim 1, further comprising a detectable marker.

4. The nucleic acid construct of claim 3, wherein the detectable marker is a fluorescent protein.

5. A lymphocyte transfected with the nucleic acid construct of claim 1.

6. The lymphocyte of claim 5, which is a human B cell.

7. The lymphocyte of claim 5, which is a Ramos human B cell.

8. The lymphocyte of claim 5, which is a human T cell.

9. A yeast or bacterial cell transfected with the nucleic acid construct of claim 1.

10. A method of producing a repertoire of polypeptides having variant sequences of a polypeptide of interest, the method comprising:
(a) culturing the lymphocyte of claim 5 in conditions that allow expression of the nucleic acid construct, wherein the lymphocyte contains the coding region of the polypeptide of interest, thereby permitting diversification of the coding region; and
(b) maintaining the culture under conditions that permit proliferation of the lymphocyte until a plurality of lymphocytes and the desired repertoire is obtained.

11. A method of producing lymphocytes that produce an optimized polypeptide of interest, the method comprising:
(a) culturing a lymphocyte of claim 5 in conditions that allow expression of the nucleic acid construct, wherein the lymphocyte contains the coding region of the polypeptide of interest, and wherein and the lymphocyte expresses the polypeptide of interest on the surface of the lymphocyte;
(b) selecting cells from the culture that bind a ligand that specifically binds the polypeptide of interest expressed on the lymphocyte surface; and
(c) repeating steps (a) and (b) until cells are selected that have a desired affinity and/or specificity for the ligand that specifically binds the polypeptide of interest.

12. The method of claim 10, wherein the polypeptide of interest is an Ig.

13. The method of claim 12, wherein the Ig is an IgL, IgH or both.

14. A kit comprising:
(a) a lymphocyte according to claim 5; and
(b) one or more containers; and
(c) a target gene expressible in the lymphocyte, wherein the target gene encodes a polypeptide of interest.

15. The kit of claim 14, wherein the target gene is a human Ig gene.

16. The kit of claim 14, wherein the target gene is an IgL gene.

17. The kit of claim 14, wherein the target gene is an IgH gene.

18. The kit of claim 13, wherein the target gene comprises a heterologous coding region and regions encoding a transmembrane domain and a cytoplasmic tail sufficient to effect display of the target gene product on the lymphocyte surface.

19. A method of restricting nuclear activity of an enzyme that modifies nucleic acids to G1 or to S-G2/M phase of the cell cycle in a host cell, the method comprising transfecting a host cell with a fusion construct comprising a nucleotide sequence that expresses the enzyme fused to a nucleotide sequence that expresses CDT1 or geminin (GEM), wherein a fusion construct expressing CDT1 restricts expression of the enzyme to G1 and a fusion construct expressing GEM restricts expression of the enzyme to S phase.

20. The method of claim 19, wherein the enzyme is CRISPR/Cas9 or CRISPR/Cas9^{D10A}.
